(19) United States
(12) Patent Application Publication
(10) Pub. No.: US 2009/0325276 A1
(43) Pub. Date: Dec. 31, 2009

(54) INTEGRATED MICROFLUIDIC ASSAY DEVICES AND METHODS

(75) Inventors: C. Frederick Battrell, Redmond, WA (US); John Gerdes, Columbine Valley, CO (US); Wayne L. Breidford, Seattle, WA (US); Jason Capodanno, Redmond, WA (US); Stephen Mordue, Los Gatos, CA (US); John Clemmens, Redmond, WA (US); Denise Maxine Hoekstra, Monroe, WA (US); Christy A. Lancaster, Seattle, WA (US); John R. Williford, Sammamish, WA (US); Patrick Maloney, Bothell, WA (US); Joan Haab, Seattle, WA (US)

Correspondence Address: SEED INTELLECTUAL PROPERTY LAW GROUP PLLC, 701 FIFTH AVE, SUITE 5400, SEATTLE, WA, 98104, US

(73) Assignee: Micronics, Inc., Redmond, WA (US)

(21) Appl. No.: 12/411,020

(22) Filed: Mar. 25, 2009

Related U.S. Application Data
(63) Continuation of application No. PCT/US2007/020810, filed on Sep. 27, 2007.
(60) Provisional application No. 60/827,186, filed on Sep. 27, 2006.

(51) Int. Cl. C12M 1/00

(52) U.S. Cl. 435/287.2

(57) ABSTRACT

Combinations of microfluidic diagnostic testing modules for simultaneous evaluations of serological and molecular biological targets are provided, and include panel testing for both antibodies (or antigens) and nucleic acid targets in one single-use device. These improvements are directed to evaluating the overall progress and activity of a pathogenic process in real time, at the point of care, not merely the presence or absence of a particular diagnostic marker, which can often be incomplete or misleading.

### CROSS-REFERENCE TO RELATED APPLICATIONS

This application is a continuation of International PCT Patent Application No. PCT/US2007/020810, which was filed on Sep. 27, 2007, now pending, which claims the benefit under 35 U.S.C. § 119(e) of U.S. Provisional Patent Application No. 60/827,186, filed Sep. 27, 2006. These applications are incorporated herein by reference in their entireties.

### STATEMENT OF GOVERNMENT INTEREST

This invention was made with government support under Contract No. U01 A1061187 awarded by the National Institutes of Health. The government has certain rights in this invention.

### BACKGROUND OF THE INVENTION

1. Field of the Invention

Point of care availability of biomolecular analysis is a critical link in extending medical care to billions of people without access to central laboratory facilities and the latest in research discoveries. Our work in microfluidics has sought to deliver products that meet those needs.

2. Description of the Related Art

Co-assigned patents and patent applications relevant to the development of clinical assays in a microfluidic device test format include U.S. Pat. Nos. 6,743,399 (“Pumpless Microfluidics”), U.S. Pat. No. 6,488,896 (“Microfluidic Analysis Cartridge”), U.S. Pat. No. 5,726,404 (“Valveless Liquid Microswitch”), U.S. Pat. No. 5,932,100 (“Microfabricated Differential Extraction Device and Method”), U.S. Pat. No. 6,387,290 (“Tangential Flow Planar Microfluidic Fluid Filter”), U.S. Pat. No. 5,872,710 (“Microfabricated Diffusion-Based Chemical Sensor”), U.S. Pat. No. 5,971,158 (“Absorption-Enhancing Differential Extraction Device”), U.S. Pat. No. 6,007,775 (“Multiple Analyte Diffusion-Based Chemical Sensor”), U.S. Pat. No. 6,581,899 (“Valve for Use in Microfluidic Structures”), U.S. Pat. No. 6,431,212 (“Valve for Use in Microfluidic Structures”), U.S. Pat. No. 7,223,371 (“Microfluidic Channel Network Device”), U.S. Pat. No. 6,541,213 (“Microscale Diffusion Immunoassay”), U.S. Pat. No. 7,226,562 (“Liquid Analysis Cartridge”), U.S. Pat. No. 5,747,349 (“Fluorescent Reporter Beads for Fluid Analysis”), US Patent Applications 2005/0106066 (“Microfluidic Devices for Fluid Manipulation and Analysis”), US2002/0160518 (“Microfluidic Sedimentation”), US2003/0124619 (“Microscale Diffusion Immunoassay”), US2003/0175990 (“Microfluidic Channel Network Device”), US2005/0013732 (“Method and system for Microfluidic Manipulation, Amplification and Analysis of Fluids”), US2007/0042427, “Microfluidic Laminar Flow Detection Strip”, US2005/0129582 (System and Method for Heating, Cooling and Heat Cycling on a Microfluidic Device); and U.S. Provisional Patent Applications US60/816,204 titled “Methods and Devices for Microfluidic Point of Care Assays”, US60/953,045 titled “Sanitary Swab Collection System, Microfluidic Assay Device, and Methods For Diagnostic Assays”, and US patent documents titled “Microfluidic Cell Capture and Mixing subcircuit”, “Microfluidic Mixing and Analytical Apparatus,” “Microscale Diffusion Immunoassay Utilizing Multivalent Reactants”, all of the above of which are hereby incorporated in full by reference. Also representative of microfluidic technologies that are co-assigned are PCT Publications WO2006/076567, WO2007/106579, and WO2007/064635, which are incorporated herein in full by reference.

Recent improvements in microfluidic diagnostic systems are due in part to advances in materials and fabrication, to the inherent rapidity of mass and heat transfer at the microscale, and to increases in detection sensitivity, but also represent a continuing effort at innovation.

In about 1992, Wilding at the University of Pennsylvania (U.S. Pat. Nos. 5,304,487; 5,486,335; 5,498,392; 5,587,128; 5,955,029; 6,953,675) filed for patents on microfabricated silicon-based devices for performing PCR. Envisaged was a family of small, mass-produced, disposable “chips” for rapid amplification of cellular or microbial nucleic acids in a sample. The devices included a sample inlet port, a “mesoscale” flow system, and a means for controlling temperature in one or more reaction chambers, where “mesoscale” refers to features, chambers and flow passages with at least one cross-sectional dimension on the order of 0.1 μm to 500 μm Heating and cooling means disclosed included electrical resistors, lasers, and cold sinks. Off-chip pumps were used to control fluid flow and to deliver reagents. Printed subcircuits, sensors on the chip, and pre-analytical binding means for trapping and concentrating analyte were suggested. The common fluid channel, which also served as the analytical channel, was used to transport cell lysis waste (such as bacteria or blood cell lysate) to an open vent or to an off-chip site. Means for detecting amplicons included, nonspecifically, DNA:DNA hybridization, either visually with fluorescent intercalating dyes or through rheological measurement, DNA binding to fluorescent probes or to diamagnetic (or paramagnetic) beads; and gel electrophoresis. Wilding's patent applications by 1994 also included antibody-based analytical microfluidic devices (as in U.S. Pat. No. 5,726,026).

The University of Pennsylvania devices were specific to solid state fabrication, with sample and reagent ports under the control of external syringe pumps. Cell lysis debris exited the chip through the PCR chamber prior to amplification, and no demonstrable mechanism for isolation of the operator from a biohazardous sample or waste was provided. Sharing of pump inlet and outlet ports from sample to sample poses an unacceptable risk for cross-contamination. Integrated devices combining immunoassays and nucleic acid assays in a single device or paired samples from a single patient in a single device were not anticipated or contemplated. Monolithic silicon also has the disadvantages of a high affinity for biological molecules, difficulty and cost of fabrication, and lack of flexibility in prototyping.

U.S. Pat. No. 6,576,459 describes a microfluidic apparatus with immunoassay and nucleic acid assay systems for detecting pathogens and importantly, for reducing the rate of false positives and inaccuracies of immunoassays in many counter-biological warfare applications. The single-embodiment apparatus, again fabricated with solid state technology, is designed with continuous sample processing capacity in immunoassay mode, and uses magnetohydrodynamic pumps instead of valves to direct fluid, substantially increasing cost and complexity. Interdigitated electrodes and diaelectrophoretic force are used to hold beads in place and to detect bead aggregation when crosslinked by target antigen when entering what is essentially a flow-through cuvette. One skilled in the art recognizes that the device is intended to be run in immunoassay mode continuously, which requires only relatively inexpensive antibody-coated beads, and when a positive event is detected, the sample is diverted to PCR for confirmation. The device is thus principally an environmental monitoring system, claiming only one PCR assay per device and reserving that for confirmation of a positive agglutination event. The apparatus again uses off-card reagent supplies and waste disposal and thus lacks critical safety features for clinical use. The apparatus also remains problematic insofar as the heat required to drive a PCR reaction is likely to irreversibly denature the antibodies immobilized in the detection apparatus.

Accordingly, although there have been advances in the field, there remains a need in the art for improved microfluidic devices for point of care applications. The present invention addresses these needs and provides further related advantages.

### BRIEF SUMMARY OF THE INVENTION

Treatment and prognosis of a pathological process, including disease, infection or other pathology, can very much depend on recognizing the correct phase and type of the process—acute versus convalescent, primary versus secondary, chronic versus opportunistic, and so forth. The problem of interpreting the relevance of laboratory diagnostics has not generally been posed this way because that has been the role of the physician. However, as the costs of laboratory diagnosis continue to decrease, and the costs of physicians increase, it is time to ask how to design combined, multifactorial laboratory diagnostic modules or panels so as to better evaluate the clinical significance of laboratory findings.

Several examples illustrate the problem. Consider Dengue Fever. In the absence of compounding factors, viremia generally clears within about a week following onset of symptoms. This often corresponds to the appearance of an IgM response in sufficient titer to neutralize the virus in blood [Lindegren J et al. 2005. Optimized diagnosis of acute dengue fever in Swedish travelers by a combination of reverse transcription-PCR and immunoglobulin M detection. J Clin Microbiol 43:2850-2855]. Thus the need for a two-pronged approach to laboratory diagnosis: early in the infection, viral particles can be detected in blood by nucleic acid assay; however, a week or so into the infection, the nucleic acid assay might be negative, but by then, serological testing for IgM will be positive. The patient may continue to be infectious in the convalescent period. Thus, combining the two diagnostic tests in a single device as provided here offers not only the assurance of a diagnosis regardless of the stage of the disease, but also additional useful information that can help characterize the progression or phase of the disease at the time the patient is examined and better ensure the public safety, an improvement over assays that merely detect the presence or absence of a molecular marker. Similarly, without differentiating IgM from IgG, detection of an antibody to Dengue in endemic areas is difficult to interpret. Corroborative evidence of viral particles is a useful supplement to antibody testing, because only IgM is diagnostic of an active infection. Also, because Dengue can be difficult to differentiate from other fever pathologies clinically, there is an unmet need for simultaneous co-assay for other agents or conditions by a dual immunological and nucleic acid approach, as would be met by febrile panel assay combining immuno- and nucleic acid assay capability, termed here a “mixed format assay panel”.

Streptococcus pyogenes, a pathogenic microorganism that can commence an infection with an unremarkable sore throat, can be diagnosed by molecular biological analysis of throat specimens or blood [Leung A K et al. 2006. Rapid antigen detection testing in diagnosing group A beta-hemolytic streptococcal pharyngitis. Expert Rev Mol Diagn 6:761-6; Pingle M R et al. 2007. Multiplexed identification of blood-borne bacterial pathogens. J Clin Microbiol 45:1927-35], but the serological evidence of an immune response is a better prognostication for the heart disease and kidney failure that are frequent sequelae to untreated or chronic infections. This organism shortens and degrades the life of almost one third of those who live without access to antibiotics. Combined laboratory testing for both active infection and serological titer provide the means to aggressively treat this disabling infection, without misuse of antibiotics. Multiplexed identification of other pathogens in the same test ensures that critical co-infections will not be missed, such as dual infections with Influenza virus or Hemophilus influenza. Because both immunological and nucleic acid assays are needed to make a full differential diagnosis of respiratory infections, there is an unmet need for a respiratory panel combining both assay types in a single disposable kit.

Similarly, skin tests for tuberculosis are largely irrelevant in endemic regions where tuberculosis is common because of the risks of severe Arthus and delayed hypersensitivity responses to tuberculin. Antibody to tuberculosis (such as the 38-kDa antigen, Antigen 60, TBGL, Kp90 and LAM) can be indicative of an active infection or prophylactic immunity, and is twice as likely to be positive in blood during infection than is PCR [Arikan S et al. 1998. Anti-Kp 90 IgA antibodies in the diagnosis of active tuberculosis. Chest 114:1253-57; Al Zahrani K et al. 2000. Accuracy and utility of commercially available amplification and serological tests for the diagnosis of minimal pulmonary tuberculosis. Am J Resp Crit. Care Med 162:1323-29]. Furthermore, sputa are notoriously difficult to collect, are highly unsanitary to process. However, without supporting evidence of a pathological process implicating the live pathogen, for example by PCR of blood, saliva or sputum, the immunological diagnosis is inconclusive. Therefore, a combination blood test for antibody and nucleic acid is highly desirable, and provides the opportunity for simultaneous evaluation of HIV, which is critical because it vastly complicates treatment of the overlying tuberculosis.

These examples do not limit the scope of the invention. As another example, assay for blood antigens can yield a more complete picture of malaria than nucleic acid assay testing or microscopy alone. Aldolase in blood is a diagnostic marker for malaria, analogous to the LDH or CPK assays used universally to diagnose the severity of coronary infarction. Malarial aldolase is readily detected by immunoassay and is released in all types of malarial infection. Interestingly, pan-specific malaria-associated LDH can also be used in comprehensive screening. Preferably an immunoassay malarial panel includes HRP2 antigen. The HRP2 antigen is included to distinguish Plasmodium falciparum and mixed infections because P. falciparum is a more malignant parasite and differs in the way it is treated. Only testing with only a pan-specific probe fails to alert caregivers to a mixed infection with P. falciparum. And when these antigens are detected side-by-side with molecular nucleic acid markers, which provides added sensitivity during certain phases of the malarial lifecycle, a very comprehensive view of the malarial status of the patient emerges. Thus the approach recommended here is advantageous in assessment of malaria and so-called tropical diseases more generally.

In short, in a world where travelers can arrive from the other side of the world in the space of a night's passing, epidemiological considerations are often useless as diagnostic tools, and rapid laboratory diagnosis is essential. Historically, physicians have observed epidemiological patterns in patient's visiting their offices, for example recognizing the onset of flu season, and were often not obligated to use laboratory diagnosis. But physicians faced with a patient having generalized malaise or fatigue, or an unpathogenomic, prodromal syndrome beginning with gastroenteritis, or the onset of a non-specific respiratory syndrome beginning with runny nose and a headache, can no longer rely on epidemiological and statistical considerations in deciding what to prescribe, and how to manage the concomitant public health risks. A shotgun approach to laboratory diagnosis is often mandatory, and a “mixed shotgun” has surprising diagnostic efficiency and an overall reduced cost to society.

These examples illustrate issues of clinical management of infectious disease and internal medicine that are not adequately addressed with current laboratory diagnostics. The decision process, whereby clinical findings are correlated in a diagnosis and treatment plan, can benefit from simultaneous information regarding the patient's immune status and the presence or absence of molecular biological nucleic acid markers, most often in “panel” form. Having redefined the problem in this way, we have conceived and designed microfluidic devices or cartridges, termed here “cards”, that are sanitary, compact, require small sample volumes, are inexpensive, and use an integrated multifactorial approach to diagnose not only the nature of the illness or pathology, but also take into account the stage of its clinical course and the inherent variability of serological and molecular test results.

In one embodiment, we have integrated nucleic acid assays and immunoassays on a single disposable card, so that the molecular diagnosis based on detection of a nucleic acid target and the condition of the patient's immune response can be analyzed simultaneously. The immunological approach can be used to differentiate historical or chronic infections from acute infections, to pick up infections where the causative agent has been largely cleared from blood, and contrastingly, the nucleic acid approach can pick up infections even in the prodromal period or in mixed co-infections, thus conferring a desirable and hithertofor unavailable synergy when made available in combination.

In another embodiment, a card that differentiates an IgG and IgM response, or an IgA or IgE response, particularly in combination with nucleic acid analyses for identifying the corresponding infectious organism directly, offers a powerful tool for managing infectious diseases and co-pathologies.

Other embodiments include an on-board “multiplex detection channel”, permitting development of panels appropriate to particular clinical situations, such as respiratory pathogen panels, sexually transmitted disease panels, fever panels, biotoxin panels, and the like. Detection means also include arrays, chromogenic endpoints, fluorescent molecular beacons with FRET, and lateral flow strips on-board the device, either in multiplex or simplex detection formats. In some embodiments, the results of these detection systems are displayed in a user friendly visual format, and in others by machine readout. In one embodiment, the user makes the selection of the tests to be performed, and the tests can be performed in parallel or in series on the card.

In another embodiment, paired samples such as blood and urine, blood and throat swab, urine and cervical swab, blood and fecal specimen, and the like are collected and tested in a single device. Qualitative molecular detection of a pathogen in a normally non-sterile sample can be difficult to assess without the synergic findings of the mixed format panels. Synergy results in deeper insight into the pathological process, as for example in detecting active shedding of viral particles, in one instance detecting not only papilloma virus but also cervical cancer markers, or detecting the presence of mixed infections, such as by Neisseria gonorrhoea and by Chlamydia trachomatis, or by Malaria and Dengue, and by detecting not only a urinary or stool pathogen or toxin but also the activation of circulating leukocytes characteristic of septicemia or toxemia. Urinary detection of bacteria is of uncertain value without a corresponding detection of proteinuria or “glitter cells”, and without quantitative pathogen counts, the mere qualitative molecular detection of a possible enteric pathogen is of uncertain diagnostic significance, no matter the symptoms, absent evidence of expression of virulence factors or host responses associated with a particular pathogen in the gut or bloodstream. An advanced device can be reconfigured in the host instrument to accommodate various specimens and testing protocols. More simple cards can be designed with valves to permit an either/or approach to testing, all at relatively low cost, as is of particular value in areas with limited access to professional services.

The microfluidic card-based assays described here target biomarkers for a wide range of clinical diagnostics, providing information not only about the identity of an infectious agent or pathological process, but also the stage and progress of the disease, thus offering the physician a real time opportunity to synchronize the correct treatment with the phase of the illness and to avoid missed diagnoses.

These and other aspects of the invention will be evident upon reference to the following detailed description and attached drawings.

### BRIEF DESCRIPTION OF THE DRAWINGS

FIG. 1 tabulates symbols used in the device schematics of the following figures.

FIG. 2 is a schematic for a microfluidic card with sanitary on-board sample processing and ELISA subcircuit.

FIG. 3 is a schematic for a microfluidic card with sanitary on-board sample processing, fixed and variable temperature thermal interfaces, simplex PCR and simplex TM-FRET analytical package.

FIG. 4 is a schematic for a card device with sanitary on-board sample processing, dual fixed temperature thermal interface, an integrated magnetics interface, and a simplex PCR subcircuit with multiplex MagnaFlow target detection package.

FIG. 5 is a schematic of a positive Magnaflow detection event depicting a two-tailed amplicon and affinity immobilization of a magnetic capture bead on a test pad.

FIG. 6 is a partial schematic of a very highly integrated second order card device with FRET molecular beacon detection, a variable temperature interface, on-board multiplex cDNA synthesis, nested PCR, and multiplex detection capability.

FIG. 7 is a schematic of a second-order integrated card with on-board sample processing, dual fixed temperature thermal interface, variable temperature thermal interface, integrated ELISA and PCR subcircuits, and a TM-FRET analytical package.

FIG. 8 is a schematic for a second-order integrated card with sanitary dual, on-board sample processing, dual fixed temperature thermal interface, hybridization detection array for nucleic acid targets, and ELISA. In one embodiment of this card, a patient's blood specimen is used for ELISA and a swab specimen from the same patient is used for nucleic acid assay. Devices of this sort can be sold as part of kits for clinical or public health services testing such as sexually transmitted disease (STD) and febrile kits).

FIG. 9 is a partial schematic for an integrated second order card having features of the above devices, and showing a detail of a multiplex ELISA subcircuit, here with dual “immunocapture” and “indirect” ELISA detectors in parallel.

FIGS. 10A and 10B are sectional views of a waste sequestration chamber with sanitary vent.

FIGS. 11A and 11B show FRET panel results.

FIGS. 12A and 12B show an immunoassay panel and an immunoassay panel result.

### DETAILED DESCRIPTION OF THE INVENTION

### 1. Definitions

The following definitions are provided as an aid in interpreting the claims and specification herein. Where works are cited by reference, and definitions contained therein are inconsistent in part or in whole with those supplied here, the definition used therein may supplement but shall not supersede or limit the definition provided herein.

Biomarker: a molecule or molecules associated with a physiological condition of health or pathology in a vertebrate. Biomarkers may include not only the proteome, genome, cytology and metabolome of the vertebrate host, but also the proteome, genome, metabolome or cytology of normal flora or pathogenic infectious agents of the vertebrate body, including bacterial, protozoan, and viral pathogens. Preferred biomarkers include antigens and antibodies and nucleic acid markers inclusive of DNA, RNA, mRNA, rRNA, and anti-sense RNA.

Test samples: Representative biosamples include, for example: blood, serum, plasma, buffy coat, saliva, wound exudates, pus, lung and other respiratory aspirates, nasal aspirates and washes, sinus drainage, bronchial lavage fluids, sputum, medial and inner ear aspirates, cyst aspirates, cerebral spinal fluid, stool, diarrhoeal fluid, urine, tears, mammary secretions, ovarian contents, ascites fluid, mucous, gastric fluid, gastrointestinal contents, urethral discharge, synovial fluid, peritoneal fluid, meconium, vaginal fluid or discharge, amniotic fluid, semen, penile discharge, chancre debris, hair with attached follicle, or the like may be tested. Assay from swabs or lavages representative of mucosal secretions and epithelia are acceptable, for example mucosal swabs of the throat, tonsils, gingival, nasal passages, vagina, cervis, urethra, rectum, lower colon, and eyes, and tampons, as are homogenates, lysates and digests of tissue specimens of all sorts. Mammalian cells are acceptable samples. Besides physiological fluids, samples of water, industrial discharges, food products, milk, air filtrates, and so forth are also test specimens. In some embodiments, test samples are placed directly in the device; in other embodiments, pre-analytical processing is contemplated.

Pathogenic condition: a condition of a mammalian host characterized by the absence of health, i.e., a disease, infection, infirmity, morbidity, or a genetic trait associated with potential morbidity or mortality. Some pathogenic conditions have etiological agents.

A panel assay is an assay designed to detect more than one target, either immunological or nucleic acid-based, in parallel or in series on a single card. Such targets may be selected from infectious disease agents, for example, including mixed panels of bacteria and/or viruses, and also host-specific targets associated with a pathogenic condition. Panel targets may include generic and species-specific targets, such as rRNA, DNA, or mRNA associated with a bacterial class, genus or species, and antibodies of the classes IgM, IgG, IgA and IgE, as well as any antigen or epitope. The microfluidic assays described here are combinations of immunological and nucleic acid panels.

Microfluidic card: is a hydraulic device, cartridge or “card” with selected internal channels, voids or other microstructures having at least one dimension on the order of 0.1 to 500 microns. Microfluidic devices may be fabricated from various materials using techniques such as laser stenciling, embossing, stamping, injection molding, masking, etching, and three-dimensional soft lithography. Laminated microfluidic devices are further fabricated with adhesive interlayers or by thermal adhesiveless bonding techniques, such by pressure treatment of oriented polypropylene. The microarchitecture of laminated and molded microfluidic devices can differ. The microfluidic devices of the present invention are designed to interact or “dock” with a host instrument that provides a control interface and optional temperature and magnetic interfaces. The card, however, generally contains all biological reagents needed to perform the assay and requires only application of a sample or samples. These cards are generally disposable, single-use, and are generally manufactured with sanitary features to minimize the risks of exposure to biohazardous material during use and upon disposal.

Lateral flow Assay: refers to a class of assays wherein target binding, aggregation or agglutination is detected by applying the target-containing fluid to a porous or fibrous matrix and observing the lateral spreading properties of the target in the porous matrix. The target will bind to ligands and be immobilized in bands or test fields. Lateral flow detection is contemplated in the devices of the present invention. In the devices shown here, where lateral flow detectors are used, the porous matrix is provided in a separate chamber on-card, is valvedly connected to an assay subcircuit, is wetted by the sample or reaction mixture at one end, whereupon wicking occurs, and is vented at the other.

Herein, where a “means for a function” is described, it should be understood that the scope of the invention is not limited to the mode or modes illustrated in the drawings alone, but also encompasses all means for performing the function that are described in this specification, and all other means commonly known in the art at the time of filing. A “prior art means” encompasses all means for performing the function as are known to one skilled in the art at the time of filing, including the cumulative knowledge in the art cited herein by reference to a few examples.

Means for detecting: as used herein, refers to a device for assessing and displaying an endpoint, i.e., the “result” of an assay or “test result”, and may include a detection channel and test pads. Detection endpoints are evaluated by an observer visually in a test field, or by a machine equipped with a spectrophotometer, fluorometer, luminometer, photomultiplier tube, photodiode, nephlometer, photon counter, voltmeter, ammeter, pH meter, capacitative sensor, radio-frequency transmitter, magnetoresistometer, or Hall-effect device. Particles, beads and microspheres, impregnated with color or having a higher diffraction index, may be used to facilitate visual or machine-enhanced detection of an assay endpoint. Magnifying lenses in the cover plate, optical filters, colored fluids and labeling may be used to improve detection and interpretation of assay results. Means for detection of particles, beads and microspheres may include “labels” or “tags” such as, but not limited to, dyes such as chromophores and fluorophores; FRET probes (including those prior art means known as “Molecular Beacons”), enzyme-linked antibodies and their chromogenic substrates, radio frequency tags, plasmon resonance, or magnetic moment as are known in the prior art. Colloidal particles with unique chromogenic signatures depending on their self-association are also anticipated to provide detectable endpoints. QDots, such as CdSe coated with ZnS, decorated on magnetic beads, or amalgamations of QDots and paramagnetic Fe₃O₄ microparticles, optionally in a sol gel microparticulate matrix or prepared in a reverse emulsion, are a convenient method of improving the sensitivity of an assay of the present invention, thereby permitting smaller test pads and larger arrays. A variety of substrate and product chromophores associated with enzyme-linked immunoassays are also well known in the art and increase the detection signal so as to improve the sensitivity of the assay. Detection systems are optionally qualitative, quantitative or semi-quantitative.

Means for amplification: include conventional means known in the art as PCR (polymerase chain reaction), rtPCR (real time PCR), RTase-PCR (reverse transcriptase-linked PCR), NASBA (nucleic acid sequence based amplification), RACE (rapid amplification of cDNA ends), LCR (ligase chain reaction), SDA (strand displacement amplification), TMA (transcription mediated amplification), TAS (transcription based amplification system), LLA (linear linked amplification), LAMP (loop mediated isothermal amplification), 3 SR (sustained sequence replication), and rolling circle amplification, as described more fully in unpublished PCT Application “System and Method for Diagnosis of Infectious Diseases” (co-assigned). These means fall generally in two general categories: thermocycling means and isothermal means.

Means for sample processing: can be “on-card” or “off-card (i.e., the latter involving pre-processing of the sample) and include filtration, liquefaction, adsorption, de-salting, digestion, sonication, ball milling, precipitation, extraction, dialysis, elution, lysis, and the like.

Means for valvedly controlling: refers to a control function executed by command of a valve, the valve comprising a check valve, pinch valve, one-way valve and the like. The control function is generally a microprocessor control function, and a digital command is converted to an analog signal at a solenoid controlling a pneumatic manifold. The programmed valve logic used in controlling assay steps is stored in ROM in the host instrument. Multiple such programs are used to run multiple assays with a single host instrument. Manually controlled valves have also been tested but are not claimed here.

Differential laboratory diagnostic finding: refers to a correlation of test results and a process of deduction that leads to a diagnosis of the cause of a pathological condition, for example by identification of an etiological agent, typically with confirmatory or supporting evidence provided from both immunoassay and nucleic acid assay test results. The process of reaching a diagnosis can be performed by a physician, for example, or can be semi-automated, using an algorithm for performing a differential laboratory diagnosis, where the algorithm comprises microprocessor-executed instructions for correlating positive test results of a multiplex nucleic acid assay for at least one pathogen with positive test results of a multiplex immunoassay for said same at least one pathogen, wherein said multiplex nucleic acid assay and said multiplex immunoassay test results are input into the algorithm by an optoelectronic device interfacing with the nucleic acid assay subcircuit and the immunoassay subcircuit of a microfluidic card of the present invention.

“Conventional” is a term designating that which is known in the prior art to which this invention relates. Unless the context requires otherwise, throughout the specification and claims which follow, the word “comprise” and variations thereof, such as, “comprises” and “comprising” are to be construed in an open, inclusive sense, that is as “including, but not limited to”. “About” and “generally” are broadening expressions of inexactitude, describing a condition of being “more or less”, “approximately”, or “almost” in the sense of “just about”, where variation would be insignificant, obvious, or of equivalent utility or function, and further indicating the existence of obvious minor exceptions to a quantity, rule or limit. The word “plurality” is taken to indicate “more than one”.

### 2. Engineering of Microfluidic Elements

The microfluidic devices disclosed here are formed from multiple subcircuits corresponding to independent assay modules, but integrated together in a single device. Each subcircuit in turn is made up of microfluidic elements or components.

Elements of these subcircuits include microfluidic channels, tees, chambers, valves, vias, filters, solid phase capture elements, isolation filters, pneumatic manifolds, blister packs (with reagent pouches), waste sequestration chambers, sanitary vents, bellows chambers, bellows pumps, optical windows, test pads, and microchannel-deposits of dehydrated reagents, optionally including buffers, solubilizers, and passivating agents. The subcircuits are generally fabricated of plastic, and may be made by lamination, by molding, and by lithography, or by a combination of these technologies.

The card devices are typically single-entry, meaning that after a sample or samples are introduced, the device is sealed so that any potential biohazard is permanently entombed in the card for disposal. The cards are typically self-contained, in that any reagents needed for the assay are supplied with the device by the manufacturer. It is understood that microfluidic devices optionally may include RFID, microchips, bar codes, and labeling as an aid in processing analytical data and that the host instrument for card docking is optionally a smart instrument and can communicate patient data and test results to a network.

Referring now to the figures, we begin with selected components that make up the microfluidic subcircuits of the invention. Table 1 recites these elements or subcombinations. Microfluidic channels (1) also termed “microchannels”, are fluid channels having variable length, but one dimension in cross-section is less than 500 um. Microfluidic fluid flow behavior in a microfluidic channel is highly non-ideal and laminar and may be more dependent on wall wetting properties, roughness, liquid viscosity, adhesion, and cohesion than on pressure drop from end to end or cross-sectional area. The microfluidic flow regime is often associated with the presence of “virtual liquid walls” in the channel. Microfluidic channels are fluidly connected by “tees” (2) to each other or to other process elements. Valves (3) are formed in microfluidic channels, and may be check valves, pneumatic check valves, pinch valves, surface tension valves, and the like, as conventionally used. Process flow direction, generally in a microfluidic channel, is indicated with an arrow, and is unidirectional (4) or bidirectional (5, reciprocating). A microchannel with valve and unidirectional process flow is symbolized in the drawings as shown (6).

The card devices generally contain an overlying pneumatic manifold that serves for control and fluid manipulation, although electronically activated valves would be equivalent. In order to reduce complexity, the drawings do not show the pneumatic manifold, but its location is made implicit by the location of air ports throughout the fluid subcircuit. Air ports (7) are connected to the pneumatic manifold, and generally activate bellows pumps. Where the valves are pneumatically actuated, air ports are also implicit, but are not shown here, again to reduce complexity by not showing the obvious. Air ports are sometimes provided with hydrophobic isolation filters (8, any liquid-impermeable, gas-permeable filter membrane) where leakage of fluid from within the device is undesirable and unsafe. Vents are indicated as shown at 9 and 10, by a concentric circle within an air port. Vents are not generally directly connected to the pneumatic manifold, but serve to equalize pressures within it.

Reaction chambers are generally indicated with a rectangular box (11), and should be considered to be inclusive of rectangular chambers, circular chambers, tapered chambers, serpentine channels, and various geometries for performing a reaction. These chambers may have windows for examination of the contents, as in detection chambers. Waste sequestration receptacles 12 are indicated by circles and have specialized structure that will be explained below. Waste receptacles are optionally vented with sanitary hydrophobic membranes.

Detection chambers 13, 14, 15, and 16 are shown symbolically, and generally combine a view window with an underlying test field or with solid-phase test pads where the progress or endpoint of the assay can be monitored, either visually or optoelectronically by any conventional detection means. Detection chambers have structure corresponding to the underlying detection technology, here corresponding to a window with test pads for heterogeneous binding (13) of antibody or nucleic acid targets, a window for solution or electrochemistry (14), a window with lateral flow strip assembly (15), and a window with hybridization array (16), illustrating common subtypes of detection systems. The droplet 17 is a universal symbol for a liquid sample of any type, and the swab (18) is a universal symbol for a solid sample or liquefied solid of any type.

### 3. Engineering of Integrated Assay Systems and Methods

Integrations of the microfluidic elements described above into fluidic subcircuits and functional biomolecular assay devices are now illustrated. A first-order integration refers to a device for either an immunoassay or a nucleic acid assay from a sample such as blood or liquefied solids, and may involve subcircuits for sample preparation, analyte extraction, immunological reaction and detection, or nucleic acid amplification and detection. A second-order integration refers to a device for both an immunoassay and a nucleic acid assay from a sample such as blood or solids or a nucleic acid assay from a sample such as blood and a second nucleic acid assay from a sample such as a throat swab. Assays of a first-order or second-order integration may be simplex or multiplex, but panel assays are preferred.

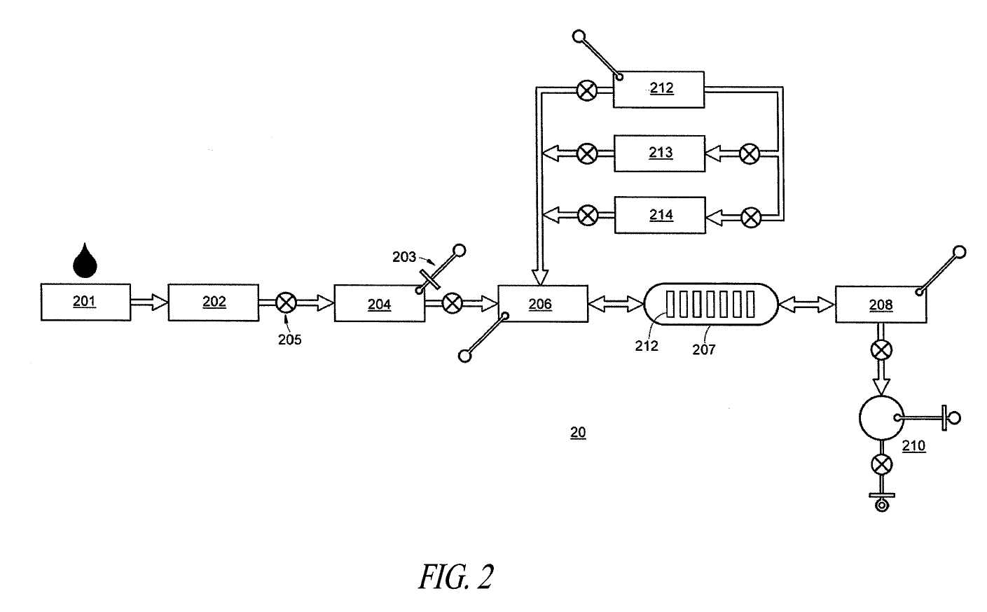

Referring again to the figures, FIG. 2 is a schematic of a first order integrated subcircuit (20) for an immunoassay. A liquid sample is introduced into a sample port (201), optionally with sample processing. Here whole blood is filtered (202) under suction to obtain plasma. Air port 203 draws negative pressure on a diaphragm in plasma collection chamber 204, which is a bellows pump with diaphragm and pneumatic actuator. The hydrophobic filter element on the air port is a safety backup in the event that the diaphragm fails. Filters such as glass fiber filters or polypropylene depth filters are suitable for separating plasma from small amounts of whole blood. Valve 205 is then closed to seal the plasma sample in the device. The contents of plasma filtrate chamber are then forced under positive pressure on the diaphragm through the pneumatic manifold into the first mixing chamber (206), which is also fitted with a diaphragm and pneumatic actuator.

In performing sandwich ELISA with the device shown here, the plasma or serum sample itself is used to wet the test pads in the detection chamber. Antibodies in the undiluted sample are captured by the antigens spotted on test pads within the detection chamber. The sample is pumped, with reciprocating fluid flow, back and forth between mixing chambers 206 and 208. When sufficiently adsorbed, the sample is discarded to waste 210, which may comprise an active diaphragm and pneumatic actuator and vent. The wash buffer pouch in its blister chamber 212, is then ruptured under positive pressure (the chamber is fitted with a pneumatic actuator), and an aliquot is used to rinse the mixing chambers and detection chamber, with mixing during each sequential wash. Rinses are passed to waste 210.

When rinsing is complete, wash buffer is used to rehydrate an enzyme-linked antibody with specificity against the target immunoglobins captured on the test pads. Conjugated antibody is contained in on-board blister chamber 213. The proper valves in the wash buffer pouch valve tree are closed and opened so that pressure on the wash buffer pouch forces buffer through chamber 213, through upstream mixing chamber 206, and into the detection chamber 207, where it reacts with any capture antibody. The paired bellows pumps (206, 208) move the enzyme-antibody mixture back and forth across the test pads to facilitate capture and binding.

On completion, the detection chamber and associated fluidic subcircuit is again purged and rinsed with wash buffer. The rinse and flush process can be repeated with reciprocal fluid flow by closing all valves to isolate the two mixing chambers, and alternately pressurizing the bellows diaphragms on each sides of the detection chamber. All rinses are discarded to waste sequestration receptacle 210.

Detection is completed by rehydrating the chromogenic enzyme substrate in blister chamber 214 and introducing it into detection chamber 207, where it will react with bound enzyme. The chromogens used are generally insoluble and precipitate on the test pads 212. A positive endpoint is indicated by the formation of a characteristic color on the test pad of interest. Labeling accompanying the optical window aids in interpretation of the visual result.

Use of the device is described in Example 4, although the protocol is modified so that antigens in blood are captured on test pads coated with immobilized reagent antibody. It should be emphasized that devices of this kind, when on-board reagents are properly formulated, can be used to detect both antibodies and antigens of the malaria parasite in blood. Malarial aldolase is an example of an antigen that can be detected by capture in an immunoassay. Assays in less than 7 minutes have been achieved for malaria in this way. Also conceived are immunoassays in which an enzyme-linked antibody is not required to detect an endpoint. Detection of selected host enzymes may be an indication of an active infection and may provide added diagnostic information, as by assay for leukocyte esterase or phosphatase, which may increase in blood during febrile reactions. Fluorophores may also be used, and can have the advantage of greater sensitivity, although requiring the device be placed in a fluorescence spectroscope. Beads are another conventional detection means for improving the detection endpoint, and are used in immuno-agglutination assays for example, as an alternative to ELISA.

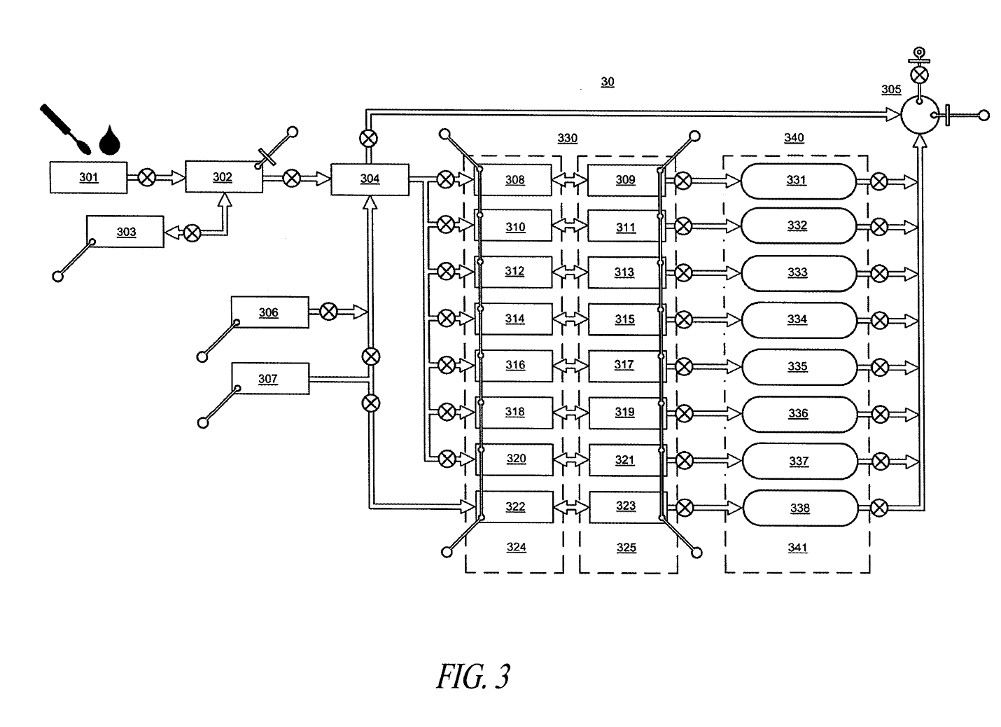

FIG. 3 is a schematic of a microfluidic subcircuit (30) for nucleic acid assay. This first order integration involves subcircuits for extraction of nucleic acids from the sample, amplification of the nucleic acids, and detection of the target sequences by TM-FRET probe. A multiplex assay is illustrated.

For illustration, anticoagulated whole blood is pipetted into the device port (301) on the left and aspirated into the lysis chamber 302. Other sample types may be processed with or without added processing. Lysis buffer is then added from lysis buffer blister chamber 303. In this embodiment, lysis buffer contains a chaotrope in combination with a detergent to reduce associations between nucleic acids and adherent molecules, and optionally contains a nuclease inhibitor and chelator such as EDTA to reduce nucleic acid degradation prior to wash.

In some instances, it may be desirable to analyze the sample for RNA species. In these cases, an inhibitor of RNAase is optionally included in the lysis buffer. We have used a modification of the Boom method (4.5M guanidinium thiocyanate, in combination with detergents such as sarcosine and Triton X-100 with weakly acidic buffer) to remove sufficient hemoglobin from whole blood so as to render the nucleic acid suitable for PCR.

In FIG. 3, note that the lysis buffer pouch chamber and lysis buffer can be isolated from the rest of the microfluidics by closing the apposing valves. Pressure and suction in the air ports over chambers 302 and 303 can then be used to cycle flow back and forth between the two chambers, facilitating mixing and lysis.

The lysate is then passed through a nucleic acid target capture assembly 304, which has reversible affinity for nucleic acids. The target capture material is generally an electropositive hydrophilic material, typically also rich in hydroxyl groups. A guide for selection of suitable target capture materials is found in U.S. Pat. No. 5,234,809, which is incorporated herein in full by reference. The target capture assembly may be, for example, a silica surface, a fiber matrix or filter composed of materials such as silica, a bed of silica or aluminum oxide beads, a fitted plug of derivatized zirconium, and the like, adapted to the dimensions, hydrostatic pressures, and flow rates of a microfluidic device. Beads may be coarse or fine, but are preferably generally homogeneous in size. Fibers may be coarse or fine, and loosely packed or tightly packed, as is required to obtain the necessary surface to volume ratio, flow rate and acceptable pressure drop. Means for sealing the bed matrix material or fiber pad to the walls of a microfluidic chamber include rabbet or mortise construction, gasket or adhesive as sealant, plastic solvent or sonic welding, pressure fit, or elements of prepackaged modular construction that can be snap fit into place so that all fluid must egress through the filter bed. For membrane filters, supporting ribs may be microfabricated by laser ablation.

Nucleic acids are retained on the capture assembly. The lysate fluids are then discarded to waste trap 305. Following discharge of the lysate into the waste chamber under control of a valve, the target capture assembly retentate is then rinsed with wash reagent from the solvent wash blister chamber 306. Wash reagent can consist of anhydrous ethanol, 70% to 95% ethanol in water, acetone, or acetone, ethanol, water mixtures, optionally with buffer. The solvent is stored on-board in a foil-lined “blister pack”, which is punctured at a programmed time under pneumatic control, so that the contents wash the target capture assembly retentate and are passed to waste 305. Wash reagent removes lipids, EDTA and salts not compatible with PCR amplification, while precipitating nucleic acids on the solid support. Generally, after the wash rinse is completed, the target capture material is briefly dried under a stream of sterile filtered air from the pneumatic manifold to remove residual solvent.

Following washing, the nucleic acid retentate is eluted from the target capture assembly with elution buffer from the elution buffer blister chamber 307. The process of purification of nucleic acid from whole blood with this subcircuitry takes less than 5 min. Serendipitously, elution in the target capture assembly shears high molecular weight genomic DNA into fragments more suitable for PCR, an added advantage in detecting low copy number targets. And because the process takes place entirely within the closed card body, there is essentially no risk of outside contamination following entry of the sample.

By using the eluate itself as the rehydration medium for the dehydrated PCR mix in the amplification subcircuits, target sequences are not further diluted. Accordingly, elution buffer, by design, can serve as PCR buffer. Elution buffer is designed to be bifunctional, and seamlessly integrates sample preparation and PCR amplification in a way not previously attempted at the microscale. The sample preparation and nucleic acid extraction subcircuit yields nucleic acids that can be used in PCR immediately, without the need for intermediate isolation (or purification), as was a drawback of earlier procedures. The elution buffer, containing target nucleic acids, is expelled into the first of the paired bellows chambers of the PCR Fluidics and Thermal Interface Assembly (330), where it rehydrates a dried “PCR mix” containing reagents, enzymes and optional FRET probes, and is heated above its denaturation point, whereupon PCR is commenced. Here, seven simplex PCR reactions are performed in parallel, using paired bellows pumps 308/309, 310/311, 321/313, 314/315, 316/317, 318/319, 320/321. The leftmost member of the bellows pump pair is heated to a temperature above the melt point of the nucleic acid targets, the rightmost of the bellows pump pair is heated to a suitable annealing temperature for PCR. Heat sources 324 and 325 on the host instrument are provided for this purpose. Bellows pump pair 322/323 are used as a negative control, and receive no patient-specific nucleic acids.

In FIG. 3, note that multiple branching, parallel “simplex” PCR reactions are performed by splitting the eluate. This is one option. Alternatively, one or more multiplex PCR reactions may be performed in parallel. Simplex and multiplex refer here to the number of primer pairs used in the PCR reaction. When only one primer pair is used in each PCR reaction, the amplification is “simplex”; when more than one primer pair is used, the amplification is “multiplex”. In FIG. 3, each of the parallel PCR reactions contains PCR mix with only one primer pair per reaction. Also anticipated in FIG. 3 is the capacity to expand the number of amplification channels, each with one or more separated primer pairs in the PCR mix, and then recombine the products for true multiplex detection at the detection station. In some embodiments, 64 or more amplification channels are provided. More preferred are 16 or fewer channels selected to perform the required differential diagnosis and present the detection event in a visually accessible form. Valves and pumps are ganged on the pneumatic manifold to simplify the command logic. In another embodiment, multiple samples can be analyzed on a single card.

It can be observed that because the PCR reactions are ported separately into separate detection chambers 331, 332, 333, 334, 335, 336, 337, and 338, the detection events are also simplex. Here, the detection chamber assembly 340 is contacted with a variable temperature thermal surface 341 external to the device, as would be suitable for FRET detection and the use of molecular beacons.

The amplification subcircuit illustrated here is designed to optimize heat exchange and mixing by recirculating the reaction mix between bellows chambers mounted on separate fixed temperature heat sources 324, 325 external to the card. Here, the fluidics interface with the heat source through a plastic film engineered for rapid heat transfer under pressure. Complete cycle times of under a minute are readily obtained. Cycle times of less than 30 sec have been routinely demonstrated. Times from sample introduction to assay result or detection event are less than 30 min, more preferably less than 20 min, and most preferentially 12 min or less. Time for PCR amplification is less than 25 min, more preferably less than 15 min, and most preferentially 10 min or less.

Optionally, a single TEC block with variable temperature control can be used for thermocycling. Whether fixed temperature or ramped temperature, control for the temperature block or blocks is generally handled off-device and is integrated with control of the pneumatics or other valves and pumps. Various means for heating and cooling are well known in the art. Heating means include conductive transfer, TEC, irradiation, and on-card resistive elements, as disclosed in the prior art.

Each FRET Detection Chamber 331-338 is used to detect FRET probe binding to the target template amplified in the adjoining PCR reaction. The FRET detection chambers are also mounted on a thermal interface, and the heat source is designed for temperature ramping. To detect a FRET signal, the amplicon products of the PCR reaction are first annealed with the FRET probe. The temperature is then ramped up to 90 to 100 C while fluorescence is monitored. A positive signal is determined by the class of the FRET probe, of which several classes are known, and the specificity of the signal is determined by matching the melt curve of the fluorescent signal with the expected melt curve of the target amplicon:probe hybrid. FRET can be performed in free solution or in heterogeneous assay.

Optionally, other sample types can be used in the device. Solid samples, such as tissue are typically fluidized either prior to analysis or in the device. The biocontent of swabs must be dissociated from the swab either in the device or prior to entry. Vegetable, mucous, fibrous, and unwanted particulate matter in the fluidized sample is preferably removed by pre-filtration through a filter, for example made of polypropylene. The nature of the buffer chosen for sample processing is dependent on the nature of the biomarkers sought in the assay. Detection of antibodies is generally incompatible with certain treatments used for extraction of nucleic acids, so as a general rule, the sample is either separated or split prior to nucleic acid extraction so that some sample is spared denaturing pre-treatment. This can be accomplished on card, or prior to sample application to the device. And alternatively, immunoassay and nucleic acid assay can be performed in senes.

Optionally, prefiltration can be used to separate the cellular and plasma components of blood. Special processing may be necessary for certain applications. The walls of elementary bodies of Chlamydia are richly crosslinked with disulfide bonds, and release of the nucleic acid contents can benefit by pretreatment with a reducing agent. Gram positive organisms and many yeasts contain cell walls resistant to chaotropes. Sonication is a useful tool for disrupting these organisms prior to nucleic acid extraction. Use of a ball mill has also been successfully used. Antibodies and antigens in tissue fluids, mucous, and intracellular vesicles may be released by a combination of Nonidet P-50 to avoid rupture of nucleii, and mucopeptidases, followed by filtration. No peptidoglycanases are currently known. However, chitinases are commercially available and are useful in disrupting yeasts and fungi where desired.

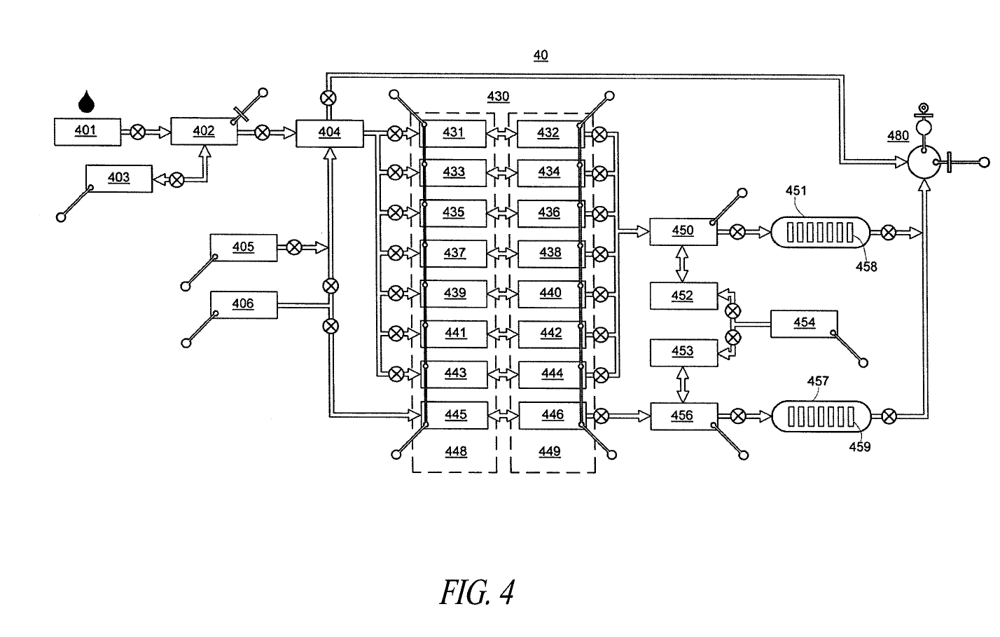

FIG. 4 is a schematic of a nucleic acid assay subcircuit 40. This first order integration involves subcircuits for extraction of nucleic acids from the sample, amplification of the nucleic acids, and detection of the target sequences by the Magnaflow process disclosed in PCT publication WO/2007/106579. Simplex PCR is illustrated with multiplex detection of targets. An illustration of an application for the device of FIG. 4 is provided in Example 3.

As shown in the FIG. 4 schematic, anticoagulated whole blood, urine or saliva is pipetted into the device at sample port 401. Events following this track those of FIG. 3 discussed above, up to the entry of sample into the detection subcircuitry. Sample is processed on card, liquefied if necessary, and aspirated into lysis chamber 402, where cellular material and aggregates are lysed and solubilized with lysis buffer from chamber 403. The lysate is then transferred to the nucleic acid target capture assembly 404, where nucleic acids are reversibly bound. These bound target analytes are first rinsed with solvent wash solution from chamber 405 and then eluted with elution buffer from chamber 406. Elution buffer is typically formulated so as to support nucleic amplification in the following step. Simplex PCR is conducted in paired bellows pumps, each pump in the pair having contact with a temperature controlled surface for denaturation 448 (leftmost) and annealing 449 (rightmost). Bellows pump pairs 431/432, 433/434, 435/436, 437/438, 439/440, 441/442, and 443/444 contain primer pairs for target analyte, bellows pump pair 445/446 is shown to indicate the use of control chambers for process validation. Here the simplex PCR products from the PCR Fluidics and Thermal Interface Assembly (430) are pooled for multiplex detection in “mag” mixing chamber 450. The control reaction shown here is collected in mag mixing chamber 456. In the Magnaflow process (see PCT Publication WO2007/106579), magnetic beads coated with avidin are used to trap two-tailed PCR amplicons tagged with a 5′-biotinylated primer. The beads are stored in dry form in mag bead reservoirs 452 and 453. The beads must first be rehydrated, as is accomplished by the valve tree and microchannels extending from the rehydration and wash buffer pouch (blister chamber 454) to the mag mixer chambers (450, 456). By placing the dried magnetic beads between these pump elements, rehydration and mixing with the incoming PCR products is initiated and is promoted by reciprocal pumping. The avidin-coated magnetic particles take up tagged amplicons (and unreacted primer).

The chambers of the “mag” subcircuit are necessarily proportioned to accommodate magnetic beads. The size of magnetic beads preferred in the assay are about 1 to 50 microns, more preferably 1 to 10 microns, and most preferentially 1.5 to 2.8 microns, mean diameter. Homogeneously sized beads are preferred. Suitable beads may be obtained from Dynal Invitrogen (Carlsbad Calif.), Agencourt Bioscience Corp (Beverly Mass.), Bruker Daltonics (Nashville Tenn.) and AGOWA (Berlin DE), for example.

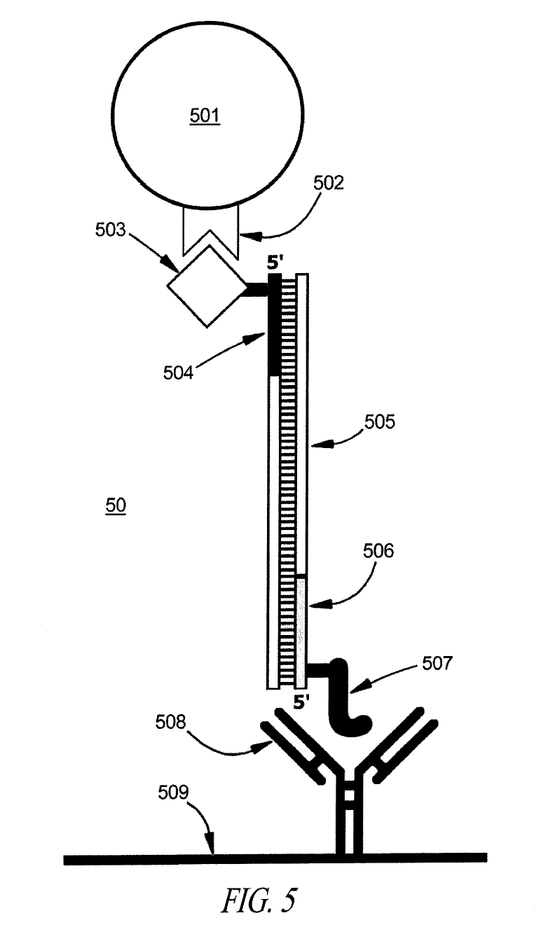

The magnetic beads, having captured biotinylated amplicons, are then transferred into the detection chamber (451) and control detection chamber (457), where multiplex test pads (indicated at 458 and 459) have been assembled during manufacture. Test pads 458, 459 contain capture antibody that will immobilize selected haptens. Each test pad antibody is unique for a particular hapten-tagged primer, i.e., the second probe used in each PCR reaction is haptenylated at its 5′ tail (thus the term “two-tailed amplicons). In the event of a positive detection event, the magnetic bead now becomes tethered to the test pad of interest (as shown in FIG. 5). Waste is pooled on board in a waste receptacle 480 fitted with sanitary vent, where it can be entombed upon disposal of the spent assay device.

A magnetic interface, not shown on the schematic, is used to manipulate the magnetic beads in chambers 451 and 457 during the detection process. Beads are fluxed back and forth in the detection chamber in close proximity to the test pads. Magnetic fields include, as is convenient, permanent magnets or electromagnets. The key point is the fact that beads are directed across and into the test pad surface by the magnetic field and provide a close encounter with the antibody or capture agent. This promotes binding interaction between the hapten and the antibody, so that binding occurs very rapidly without the need for extended incubation. The nature of the positive binding complex will be explained in more detail in FIG. 5. Once bound, the magnetic field is turned off and the test pads are readily washed to remove residual unbound particles. In practice, the time from amplification to test results is less than 4 minutes.

Rinses of the detection chamber are performed by expelling more wash buffer through the mixing chamber. Only immobilized magnetic beads are not washed into waste. Upon completion, positive detection events are characterized by a clear optical signal of the molecular complex 50 pictured in FIG. 5, which shows a paramagnetic bead 501 coated with avidin (502, or other ligand binding molecule) bound to biotin (503, or other ligand such as digitonin), where the biotin 503 is covalently bound to a first primer of an amplicon (504, biotinylated forward primer). The amplicon is tagged at its second end with a hapten (507) of a hapten-tagged reverse primer (506), which is captured by an immobilized anti-hapten antibody (508) on a solid substrate or test pad (509). Unreacted antibody test pads are clear or uncolored, whereas reacted antibody test pads are dark colored, due to the magnetic beads, and can be photographed for a permanent record. A sufficient number of immobilized beads, as present in a few microliters of reagent, result in a visual coloration of the test pad. As would be obvious to one skilled in the art, magnetic beads can be prepared with labeling aids such as QDots, dyes, RFIDs, etc, so as to be detectable when immobilized on the respective test pads. The illustration depicts a biotinylated forward primer. Note that the identity of the forward and reverse primers can be interchanged.

Returning to FIG. 4, the waste sequestration chamber 480, where all discarded reagents are trapped, is isolated from the exterior of the device by a series of elements. First, liquid reagents are absorbed in a bibulous pad, which may contain dessicants and disinfectants. The pad will freely swell as it imbibes liquid, displacing a deformable or elastic film that separates it from a vent to the outside atmosphere, through which displaced air egresses the device. Moreover, the vent itself is protected with a hydrophobic gas-permeable, liquid impermeable membrane, so that even in the event of failure of the isolation measures of the waste chamber itself, a final protective barrier is in place.

Again, samples other than whole blood may be used. A prefilter, placed between the sample port and lysis chamber, is used to clean up unwanted vegetable matter, fibers, clots, inorganic solids, keratin and the like. Provisions for processing of liquefied extracts of swabs, tampons, tissue, or scrapings, with entrained biomarkers are provided.

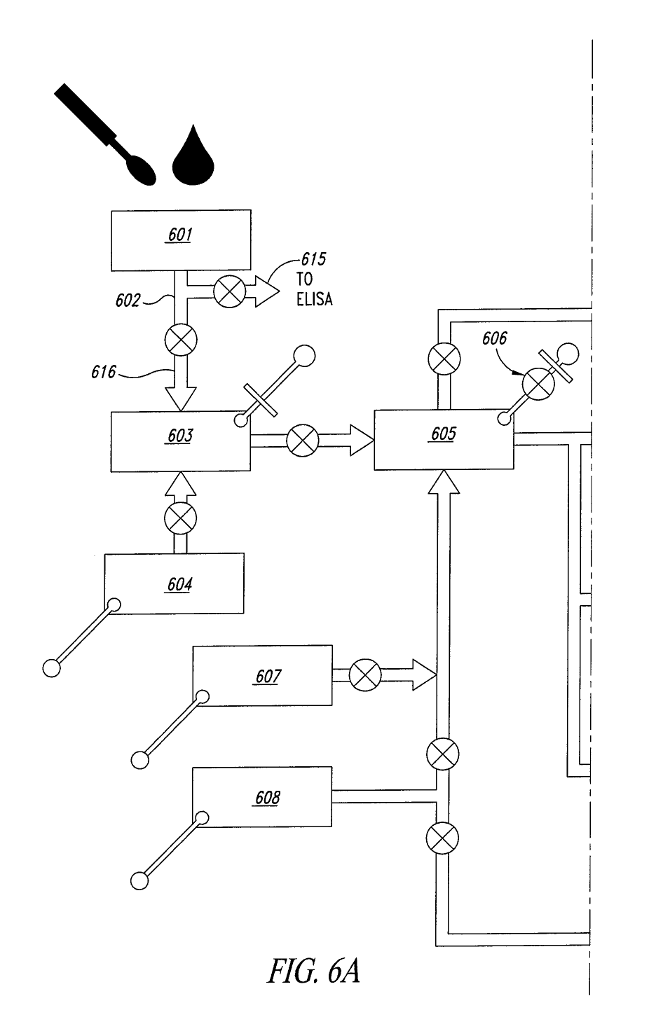

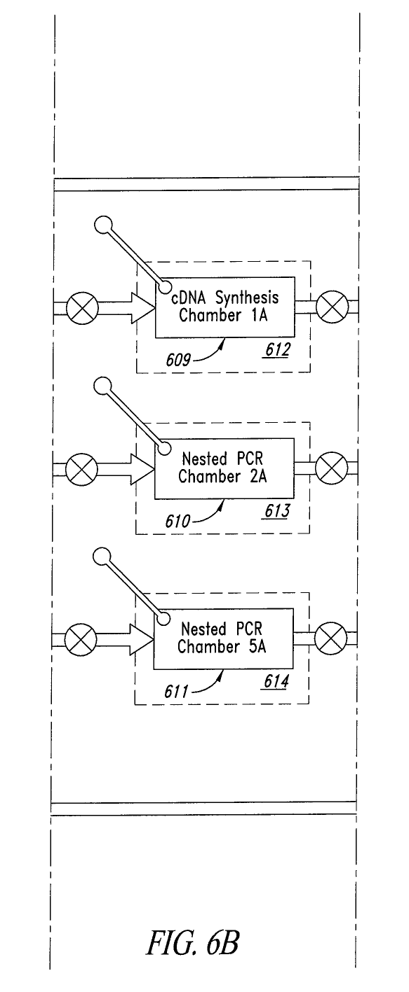

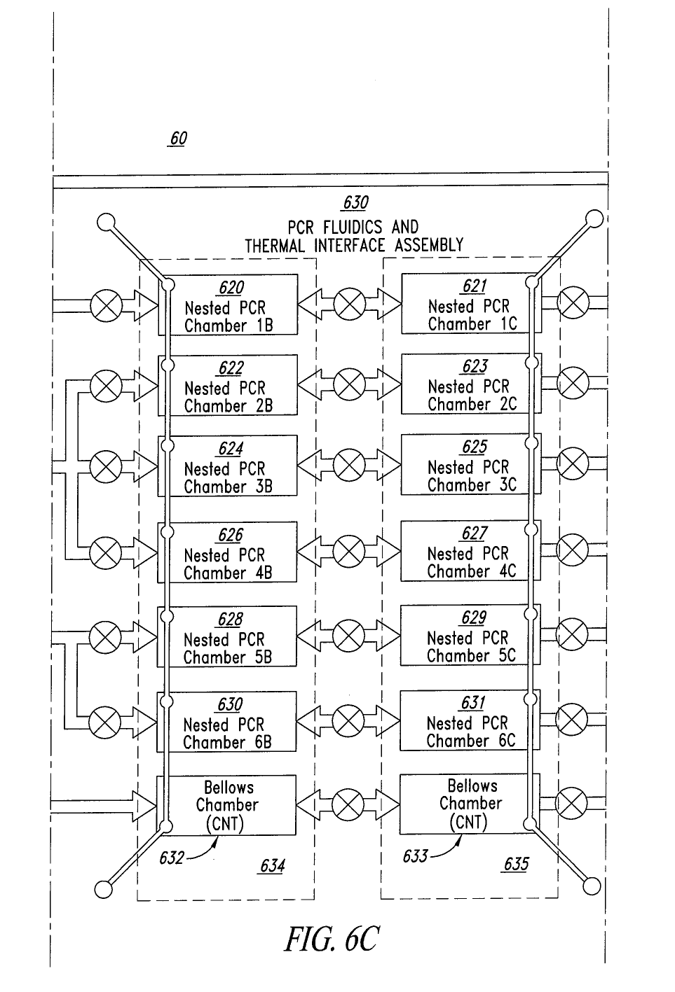

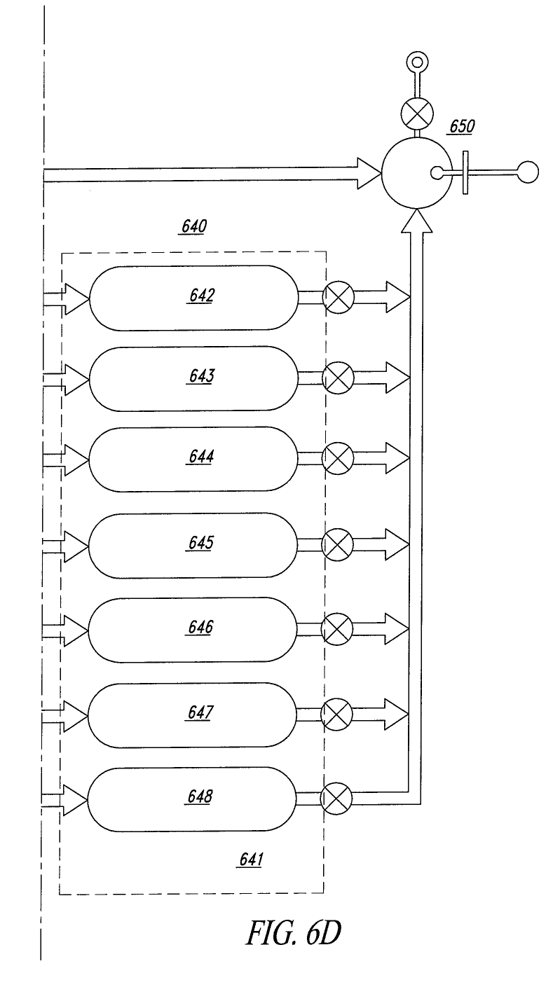

FIG. 6 is a schematic of a second order integrated subcircuit 60 for testing of nucleic acid targets that include single stranded sense and antisense RNA targets, mRNA, rRNA, and double stranded DNA, with an option for reverse transcriptase mediated cDNA synthesis, multiplex nested, sequential or asymmetric primer amplification prior to simplex PCR and simplex or multiplex detection. Note the use of one or more variable temperature thermal interfaces. An illustration of the use of a device of this kind is described in Example 11 (sexually transmitted diseases panel selected from Chlamydia trachomatis/Neisseria gonorrhoea/Trichomonas vaginalis/Mycoplasma genitalia/Papilloma Virus/Herpes simplex Type II and HIV).

Liquefied sample entering sample port 601 is divided at tee 602 into branches 615 and 616, with a portion of the sample entering an immunoassay (615, TO ELISA) such as the one shown in FIG. 2. The remaining sample enters lysis chamber 603 through branch 616 and is treated with lysis buffer from chamber 604, prior to transfer to the nucleic acid target capture assembly 605 when aspirated by air port 606. The lysate is first treated with solvent from solvent wash pouch 607 and the retentate, which is enriched in single and double stranded nucleic acids, is then eluted by buffer from the elution buffer pouch 608. The eluate is split in a branching tee network into a cDNA synthesis chamber 1A (609), and two nested PCR chambers 610 and 611. cDNA synthesis chamber 609 includes an external variable temperature interface 612, and dried reagents, and is used for reverse transcriptase-mediated synthesis of DNA from rRNA, mRNA or antisense RNA. The resultant first strand cDNA is transferred to a pair of bellows pumps 620, 621, which are part of PCR Fluidics and Thermal Interface Assembly 630. Use of paired bellows pumps and reciprocating flow to promote mixing or to do PCR is a recurrent theme in the invention. Here the leftmost bellows pump 620 is used for denaturation of DNA and the rightmost bellows pump 621 is used for annealing. Primer extension occurs at intermediate temperatures. Primer and other PCR reagents are placed in the bellows pump before sealing the device during manufacturing. Optionally, the cDNA mixture is diluted with PCR buffer. Alternatively, as shown for nested PCR chambers 610 and 611, PCR is first performed with one set of primers, and then with another. Chambers 610 and 611 are provided with variable temperature interfaces 613 and 614. In the first stage, chambers 610 and 611 serve for annealing, and chambers 622, 624, 626, 628, 630 and 632 serve for denaturation, thus forming a PCR subassembly. Note the valves between chambers 613 and 622 and between chambers 622 and 623. For stage two of nested PCR, chamber 622 serves for denaturation and chamber 623 for annealing, and so forth. The valve tree structure and bifurcating parallel paths isolate different primer sets from each other, permitting fine genetic mapping of target DNA. This also permits use of RNAase in some pathways, but not in others, for example. Genomic DNA and cDNA may be differentiated in this way, as can be useful in differentiating active and inactive retroposons.

As will be discussed in more detail in Example 141, the fluidic assemblies can also be used to subject cDNA from chamber 609 to further amplification and analysis by aspirating its contents into chambers 610 and 611. Thus, the basic elements of the microfluidic subcircuits described here can be reasserted to produce complex integrated functions specific for individual assay panels.

Bellows pump pairs 620/621, 622/623, 624/625, 626/627, 628/629, 630/631, and 632/633 (the latter in use as a negative control) are all part of PCR Fluidics and Thermal Interface Assembly 630, which is contacted with external heating elements. Temperature interfaces 634 and 635 can be fixed temperature or variable temperature controlled. Very thin polymer films, such as Mylar, nonetheless provides excellent heat transfer for volumes typically in the range of 5 to 100 uL. Control and actuation of the bellows pumps is shown here under pneumatic control in ganged manifold array. All valves in this device are pneumatically actuated under control of an external microprocessor and docking apparatus, part of the host instrument in which the microfluidic card devices are fitted or “docked” during the assay.

Upon completion of PCR, the reactants are transferred to the TM-FRET Detection Chamber Array 640, which is contacted with an external variable temperature interface and controller 641. Typically the annealed probes light up under fluorescent excitation immediately, and as the temperature in the detection chambers is ramped up, a distinctive melt curve confirmatory of the PCR product can be recorded. Detection chambers 642, 643, 644, 645, 646, 647, and 648 provide multiple simultaneous endpoints. Added parallel processing is readily achieved, including both positive and negative controls as required.

All waste is routed fluidically to a waste chamber or receptacle 650 fitted with pneumatic diaphragm, actuator and vent. These assemblies may also contain bibulous material in which waste liquids are trapped and entombed after use of the device.

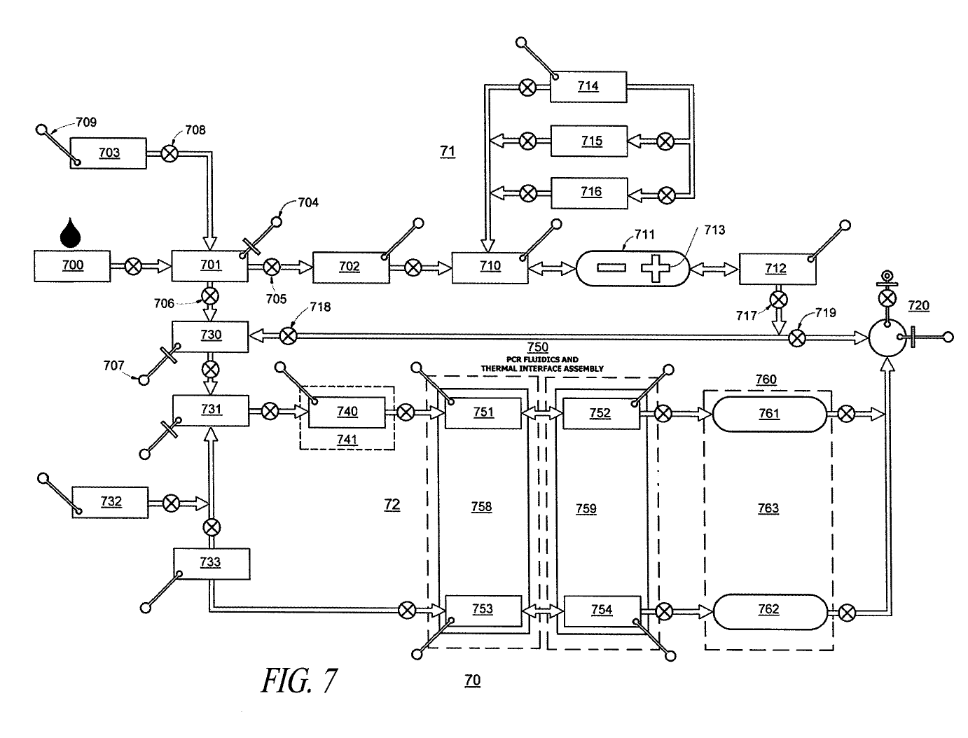

FIG. 7 is a schematic for a second-order integrated device 70 combining ELISA (71) and PCR (72) subcircuits. ELISA and PCR subcircuits are separately gated with valves 705 and 706 but share common elements 700,701 for sample processing. It should be appreciated that parallel subcircuitry may be added to increase the number of targets detected and the device complexity, as was demonstrated in previous figures. Blood entering the device at sample port 700 is immediately subjected to filtration at filter 701 by aspiration as actuated by air port 704, and a plasma fraction is pulled through into plasma filtrate chamber 702, the start of the ELISA fluidic subcircuit 71. By use of a polypropylene filter element, nucleic acid retention is minimal. When plasma filtrate chamber 702 is full, valve 705 is closed and the remainder of the sample is aspirated into lysis chamber 730.

Starting with the ELISA subcircuit 71, we follow plasma from chamber 702 into bellows chamber 710. Plasma is pulled back and forth across immunobinding sites + (713) and − in detection chamber 711 by the reciprocating action of bellows chambers 710 and 712. During this procedure, the plasma sample is isolated from the remaining subcircuits of the device. But following binding, excess plasma is redirected to join with the remaining sample in lysis chamber 730 by the action of valves 717, 718 and 719, improving sensitivity proportionally by plasma recycling. The microfluidic channel connecting chamber 712 and 730 is a plasma recycling subcircuit. While a plasma recycling system is shown here, it should be apparent that the ELISA and PCR subcircuits may also be interconnected in series to achieve the same effect. Performance of immunoassay and nucleic acid assay in subcircuits connected in series will also relieve the need to thermally insulate the serological reactions from the heat associated with thermocycling in for example, PCR reactions.

The test pads 713 (here only two are shown for simplicity) are then washed to remove nonspecifically bound ligand with wash buffer from wash buffer pouch chamber 714. The first wash can also be recycled for nucleic acid assay or is directed to waste by opening valves 717 and 719. Typically several serial washes are performed. ELISA is then completed by adding enzyme-linked anti-immunoglobin (here an antibody is being detected) and then the corresponding chromogenic enzyme substrate. Enzyme-linked anti-immunoglobin is stored on card in either dried or liquid form in chamber 715 and enzyme substrate in chamber 716. Following color development, waste may be directed to waste chamber 720, although this is not necessary because the waste is captive between valves 705 and 717.

In lysis chamber 730, lysis buffer is then added from lysis buffer pouch 703, lysing and solubilizing cellular material and debris in the sample. Lysis buffer is stored in a co-laminated plastic foil pouch under conditions that optimize its stability. Its release is controlled by the opening of a valve 708 to establish a fluid interconnection to the cell separation filter and by pressurization at air port 709 on the lysis buffer pouch in its blister pack sufficient to rupture the pouch and force or draw the contents into the lysis chamber 730. Note that the filter membrane of filter assembly 701 is also treated with lysis buffer in order to improve yield—many pathogens are localized to the cellular fraction of blood. Air ports 707 and 704 are used to generate reciprocating flow between chambers 730 and 701, which are pumping chambers. The lysate is then transferred to nucleic acid target capture assembly 731. Target capture and purification is preferably performed on a solid support with affinity for single and double stranded nucleic acids, and solvent wash pouch 732 is opened to rinse away unbound material to waste 720, reversing the earlier direction of flow in the plasma recycling subcircuit. Eluate released from the nucleic acid target capture assembly 731 by elution buffer from chamber 733 is then transferred into cDNA synthesis chamber 740, which is contacted with an independent temperature controlled interface (741, generally set at about 37° C. for reverse transcriptase mediated cDNA synthesis). Chamber 740 is provided with the appropriate biologicals to support reverse transcription of rRNA, mRNA, and anti-sense RNA as may be required. Multiple such chambers may be provided for simplex reverse transcriptase synthesis, or a multiplex approach with multiple primers may be used.

Paired bellows chambers 751 and 752 and control bellows chambers 753 and 754 make up the PCR Fluidics and Thermal Interface Assembly 740, which contacts two external temperature interfaces 758 and 759, the temperature of which is controlled by the host instrument, providing heat and heat sink functions needed for PCR. The reverse transcriptase products are first transferred into chamber 751 for denaturation, and then to chamber 752 for annealing. Following PCR, reaction products from bellows chamber 752 and control bellows chamber 754 are pumped into detection chambers 761 and 762. Amplicons can be detected by FRET, with confirmation by melt curve using variable temperature interface 763, or by other means, including but not limited to array hybridization with fluorophore-tagged primers, integrated lateral flow strips as described in USPA US2005/0013732 (“Method and system for Microfluidic Manipulation, Amplification and Analysis of Fluids”, co-assigned), Magnaflow as described in PCT Publication WO2007/106579 (“Integrated Nucleic Acid Assays”, co-assigned), and by other conventional means. Lateral flow detection can be multiplex or simplex.

Programmable valve logic choreographs these fluid transfers at every stage of the assay. PCR can be multiplex or simplex. Other conventional nucleic acid amplification systems, such as NASBA, may be substituted for PCR with appropriate restructuring of the nucleic acid assay subcircuit. In this embodiment, the fluid movements are choreographed by a pneumatic control sequence. Pneumatic signals are sent to valves, or directly raise or lower diaphragms in bellows chambers, transmitting positive or negative pressure to the fluid while keeping the sample isolated. Note that the waste chamber 720 contains the only external vent on the device, and this vent is sealed by a hydrophobic liquid-impermeable, gas-permeable membrane to prevent loss of biologics from the card.

Other sample types may be used in the device of FIG. 7. Samples containing solids may require fluidization and pre-filtration on-card or off-card. Blood, saliva and urine, all of which contain antibodies, generally require no off-card pre-processing.

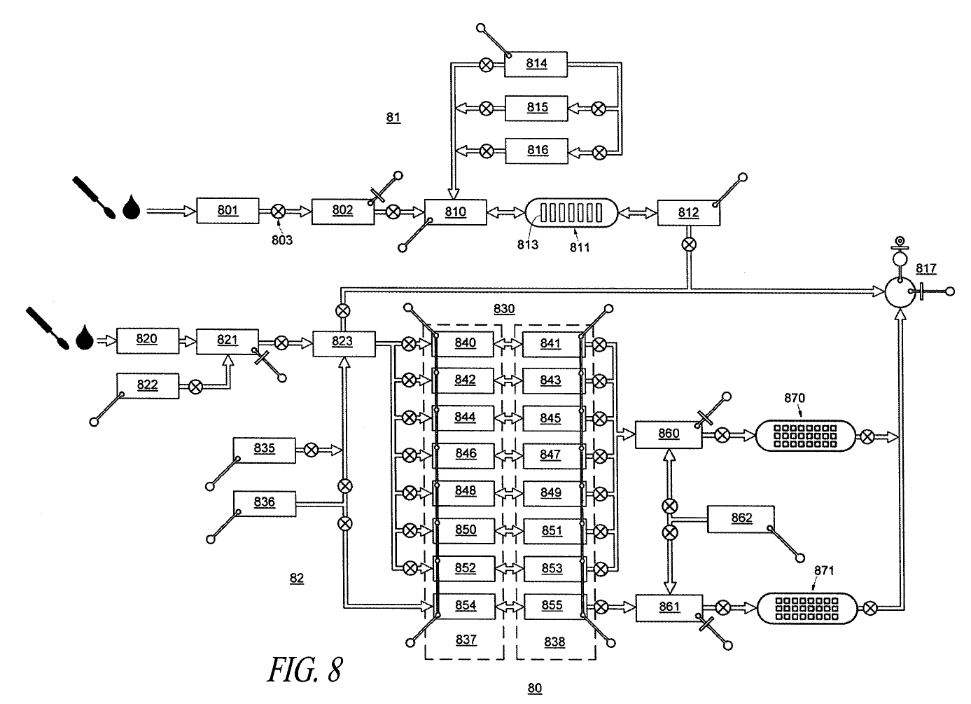

FIG. 8 is a schematic for a second-order integrated device 80 combining ELISA (81) and PCR (82) subcircuits, but the sample for ELISA can be blood and the sample for nucleic acid analysis can be a swab. In this figure, the blood sample is subjected to filtration at filter 801 by aspiration, and a plasma fraction is pulled through into the ELISA fluidic subcircuit. When plasma filtrate chamber 802 is full, valve 803 is closed. We follow the plasma from chamber 802 into bellows chamber 810. Plasma is pulled back and forth across immunobinding sites (813, test pads) indicated in detection chamber 811 by the reciprocating action of bellows chambers 810 and 812, promoting immunobinding. The test pads 813 are then washed to remove nonspecifically bound ligand with wash buffer from wash buffer pouch chamber 814. ELISA is then completed by adding enzyme-linked anti-antigens (here one or more antigens is being detected) and then the corresponding chromogenic enzyme substrate. Enzyme-linked anti-antigen is stored on card in either dried or liquid form in chamber 815 and enzyme substrate in chamber 816. Following color development, waste is directed to waste chamber 817.

A swab sample is introduced into sample processing unit 820 and a liquid extract is transferred by suction to lysis chamber 821. Alternatively, the swab-collected biological material may be processed at the bench before transfer to the device. In lysis chamber 821, lysis buffer is then added from lysis buffer pouch 822, lysing and solubilizing cellular material and debris in the sample. The lysis chamber generally includes a filter to avoid downstream clogging. Target capture is then preferably on a solid support 823 with affinity for single and double stranded nucleic acids, and solvent wash pouch 835 is opened to rinse away unbound material to waste 817. The waste chamber is vented with a sanitary hydrophobic filter 818. Eluate released by elution buffer from chamber 836 is then transferred into branched, parallel, paired bellows chambers 840/841, 842/843, 844/845, 846/847, 848/848, 850/851, and 852/853 of the PCR Fluidics and Thermal Interface Assembly 830, which contains two external temperature controlled surfaces 837 and 838 providing thermal heatsinks and control needed for PCR. Bellows chambers 837 and 838 are shown here as a mock PCR negative control. Following PCR, reaction products from bellows chamber 841, 843, 845, 847, 849, 851, and 853 are pumped through a valve into a reaction chamber 860 for mixing with hybridization buffer from chamber 862 and for denaturation, and then into detection chamber 870 with hybridization array. Control reaction chamber 861 and array 871 is also provided, and is here shown as a negative control. Detection strategies involving fluorescent primers, probes, and arrays are well known.

This device has application in detection of targets not commonly found in blood but where a blood antibody titer is expected. This class includes for example tuberculosis, where blood antibody and sputum culture are the current best practice laboratory tests. And is also applicable to Streptococcus pyogenes or Bordatella pertussis, where blood antibody appears quickly in infection, but the presence of organism in a throat swab is more indicative of infectiousness and the need for quarantine or other public health measures. Also of interest is a special case where broad shotgun screening is required, and some infectious agents are better screened in blood but others more likely detected by swab, and both sample types are to be assayed by parallel nucleic acid amplification and detection on a single device. Thus the use of two separate samples for a single patient on a single diagnostic card.

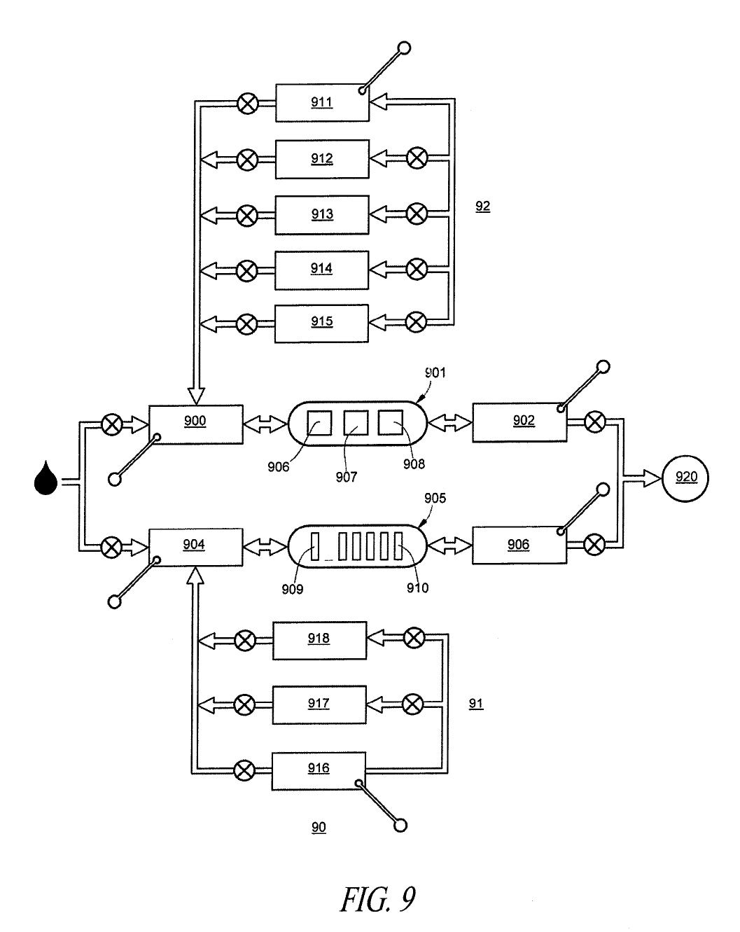

FIG. 9 is a detail of a schematic for a second order integrated device having features of the above devices, but showing a detail of a multiplex ELISA subcircuit (90), here with two “immunocapture” and “indirect” ELISA detectors in parallel (91, 92). In the schematic, plasma, or serum, entering the ELISA subcircuit 90 is split by aspiration at a tee and enters parallel, paired bellows chambers 900/902 and 904/906 separated by detection chambers 901 and 905, respectively. The upper detection chamber 901 is used to identify the class of antibody of interest in an immune response; the lower detection chamber 905 is used to identify the serovar of the infectious agent. The paired bellows pumps serve to mix the sample and induce immunobinding on test pads in the detection chambers. Upper detection chamber 901 contains negative control test pad 906, anti-human IgG test pad 907 and anti-human IgM test pad 908. Lower detection chamber 905 contains negative control test pad 909 and five viral group or serovar antigen-coated test pads (indicated at 908).

Determination of antibody class in subcircuit 92 is made as follows. Anti-human IgG and IgM test pads (907, 908) are used to collect antibody in the sample. IgA and IgE could be collected also. Wash buffer supplied from wash buffer pouch 911 is used to rinse unbound plasma proteins to waste 920. Pooled viral antigens 912 are then added to the detection chamber and bind if patient antibody is present. Mouse anti-viral antigen IgG is added from chamber 913, forming a sandwich, which is then detected with enzyme conjugated anti-mouse IgG hybridoma antibody from chamber 914 and chromogenic enzyme substrate from chamber 915.

Determination of viral antibody specificity in subcircuit 91 is made as follows. Plasma is incubated with test pads in detection chamber 905 so that viral group or serotype-specific antibodies bind to specific group or serotype-specific antigen bound to the test pad. Following antibody capture, wash buffer from chamber 916 is used to wash to waste 920 any unbound material. Enzyme-linked goat anti-human antibody from chamber 917 is used to detect the captive antibody with chromogen from chamber 918 by a typical ELISA protocol.

An application of this device is provided in Example 9. The utility of identification of the class of antibody in the immune response has multiple applications. Viremia, as another example, in Dengue Fever, generally clears within about a week following onset of symptoms. This corresponds to the appearance of an IgM response in sufficient titer to neutralize the virus in blood. Thus the need for a two-pronged approach to laboratory diagnosis. Early in the infection, blood virus particles can be detected by PCR or nucleic acid assay. A week into the infection, the nucleic acid assay may be negative, but serological testing for IgM will be positive. Note that in endemic areas, IgM must be differentiated from IgG in order to make a meaningful differential diagnosis from the laboratory data. Thus combining the two diagnostic tests in a single device provides not only assurance of a diagnosis, but also additional useful information regarding the course or state of the disease.

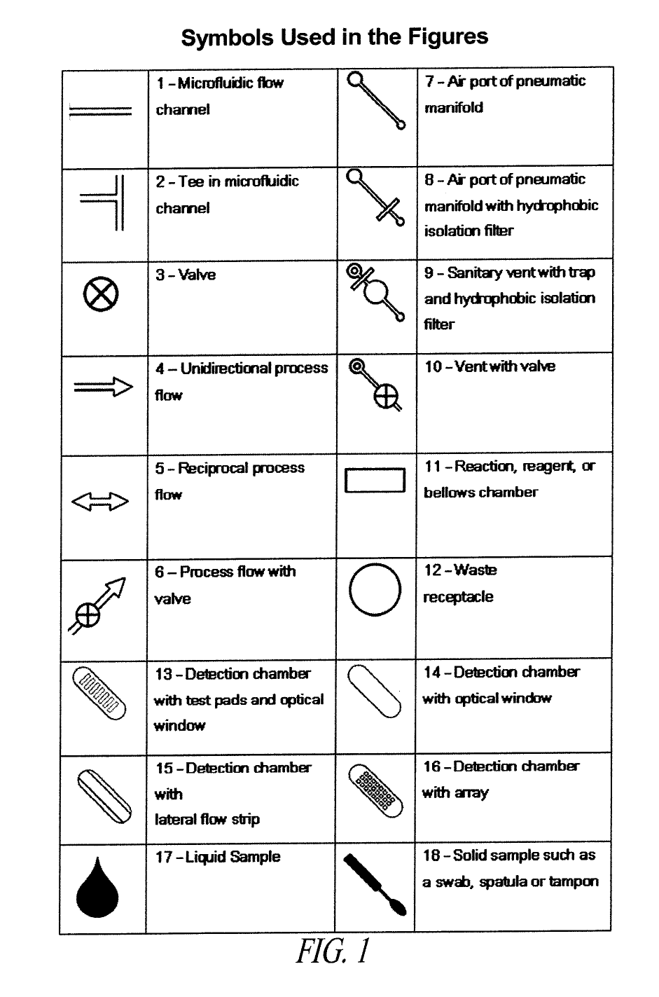

Since IgM is the hallmark of an early immune response, and IgG can result in false positives due to its persistence after active infection is over (in other words an IgG titer may represent a historical infection of no immediate clinical significance), an immunoassay that does not differentiate IgM from IgG may result in what are essentially, in terms of clinical relevance, false positives. An alternate approach to this issue is to neutralize any IgG titer in the assay so that the assay is specific for IgM. This can be accomplished by adding, for example, goat anti-IgG (Fc class specific) prior to performing ELISA. In the devices of the present invention, the addition of goat anti-IgG is a function that can be accomplished in the sample preparative elements of the device. Goat anti-IgG can be deposited in dried form in the plasma filtrate chamber (see FIG. 1 prior to entry into the subcircuit described in FIG. 9), an alternative way of differentiating IgG from IgM in the assay for Dengue Fever. This dual ELISA subcircuitry for antigen testing (91) and antibody testing (92) or antibody class testing (91) and antibody specificity testing (92) is integrated with parallel nucleic acid subcircuitry on a single device (90, FIG. 9). Whole blood is a preferred sample type. A shared wash buffer pouch can be used in this type of assay.

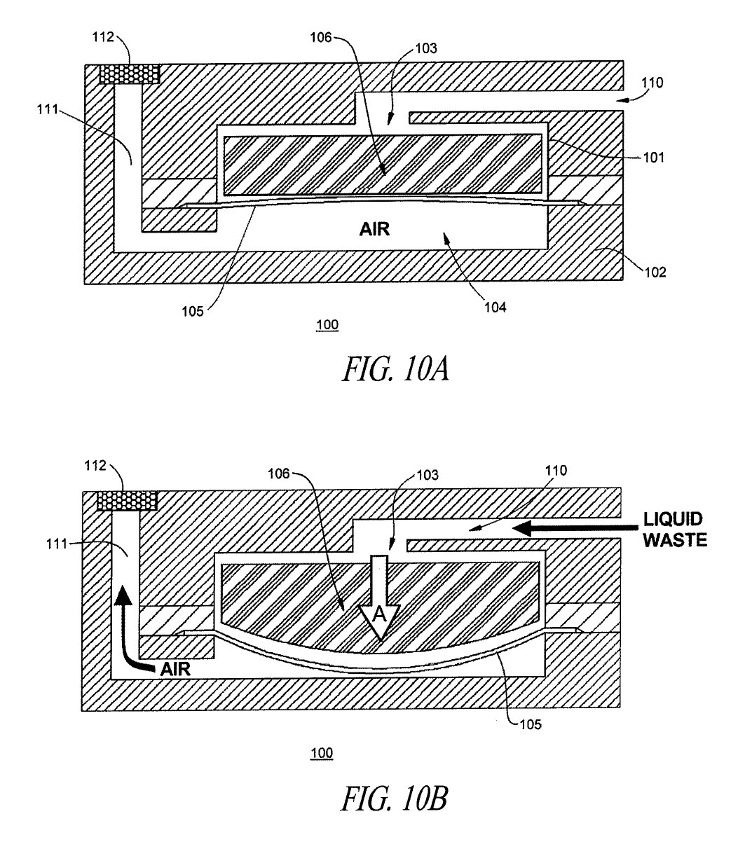

Turning now to FIG. 10, in the design of these devices, double layers of protection are frequently provided so that contaminated waste or reagent does not leave the device. FIG. 10A shows a waste sequestration receptacle 101 in a plastic body 102. The waste chamber is divided into an upper waste chamber 103 and a lower waste chamber 104 by a flexible diaphragm 105. Upper waste chamber 103 contains an absorbent pad 106 and is connected to the analytical subcircuitry of the microfluidic device by waste line 110. The waste receptacle subassembly also comprises a vent line 111 capped by a hydrophobic liquid-impermeable, gas-permeable membrane 112. The vent is typically valved, the valve comprising a check valve, pinch valve, pneumatic valve, one-way valve, and so forth, as dictated by the functional roles of the valve in the design. Several layers of the plastic body 102 are shown to generally indicate laminated construction, although a molded body construction is equivalent. A simplified waste sequestration receptacle, comprising only chamber and vent, is also contemplated. The vent is optionally sealed against liquid egress by a hydrophobic gas-permeable membrane seal.

As illustrated in FIG. 10B, fluid waste entering the waste sequestration receptacle or chamber 101 through waste line 110 encounters a fibrous, bibulous pad 106. As the pad swells (as illustrated conceptually by arrow “A”), it displaces the elastomeric diaphragm or deformable film 195 that isolates it from the outside vent (111, 112). In the event of failure of the deformable film, a hydrophobic filter mounted in the vent via stops fluid leaks.

Absorbent Pads (106) are made of materials similar to those found in absorbent articles such as disposable diapers. The absorbent core typically includes a fibrous web, which can be a nonwoven, airlaid web of natural or synthetic fibers, or combinations thereof. Fibrous webs used in such absorbent articles also often include certain absorbent gelling materials usually referred to as “hydrogels,” “superabsorbent” or “hydrocolloid” materials to store large quantities of the discharged body fluids. These materials absorb through capillary or osmotic forces, or a combination of both (see U.S. Pat. No. 4,610,678 and 5,906,602, herein incorporated by reference). The bibulous diaper or pad of fiber material is optionally treated with a dessicant. Fiber pads are typically cellulosic. Dessicants include calcium sulfate, gypsum, calcium chloride, and silica gel. Other materials include papers, sponges, diaper materials, Contec-Wipe™ (Contec, Spartanburg S.C. USA), for example.

All gas displaced as the liquid reagents are introduced into the microfluidic assay channel exits the device through a sanitary vent 111 with filter 112 which is hydrophobic and permeable to gas but not liquid, thus protecting the operator to exposure to biohazards.

Guanidinium salts, alcohol, and detergents function in the waste chamber as disinfectants. Povidone Iodine may be used in combination with the above. These disinfectants are optionally impregnated in absorbent pad 106 or can be provided as a pouch to flood the card after use. With careful design, the microfluidic device can be discarded after use without special precautions because the waste is entombed and disinfected inside the card.

The microfluidic devices of this invention have been engineered so that once a sample is placed in the device, further exposure of the operator to its contents is avoided. The design ensures a single-entry, disposable device for medical testing. Once inserted into the device, the sample port is closed and sealed, capturing the medical waste in the device, wherein which it will be entombed. Lysis buffer is also a disinfectant, and by use of a closure to seal and lock the sample port after entry of the sample, flushing the internal surfaces of the portal with lysis buffer substantially reduces the risk of accidental exposure.

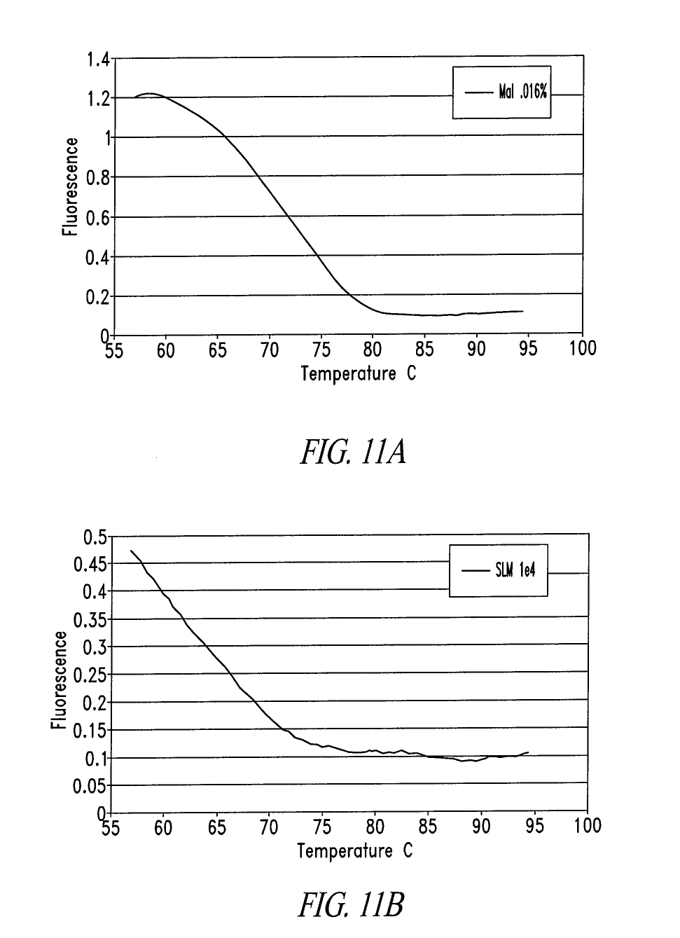

FIG. 11 shows test results and thermal melting curves for amplicon:molecular beacon complexes of Plasmodium falciparum and Salmonella paratyphi as part of a fever panel with parallel testing. Clinical blood samples were used in the assay. Nucleic acid purification, simplex PCR amplification and FRET detection were performed on a microfluidic card of the present invention. Primer:probe mixtures were obtained from Nanogen, Bothell Wash. Plotted are raw melt curves for the PCR product amplicon:molecular beacon complex with fluorescence excitation. Note that the FRET signal is present immediately upon entry into the detection chamber and that temperature ramping was carried out from about 55 to about 95 C. FIG. 11A is the plot for Plasmodium falciparum; FIG. 11B is the plot for Salmonella paratyphi. The raw curves were also replotted as second derivative to obtain a standardized melt temperature of the amplicon:probe complexes.

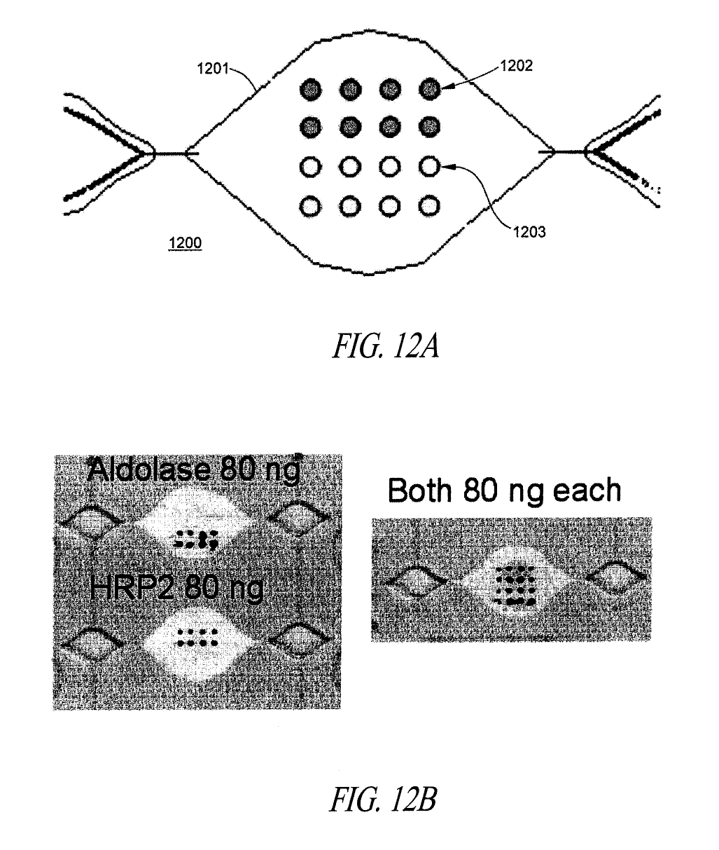

FIG. 12A is a drawing showing an arrangement of test pads in four rows (four dots per row) within an immunoassay detection chamber 1201, where the upper two rows and the lower two rows were spotted with antibodies against different malarial antigens. For the accompanying photomicrographs, row 1202 was spotted with pan-specific anti-aldolase and row 1203 was spotted with anti-HRP2 specific for Plasmodium falciparum and on-card assay with sandwich ELISA was used to detect mixed malarial antigens. Capture antibodies were obtained from Immunology Consultants laboratory, Newberg Oreg. Antigen was also purchased. ELISA was performed with HRP-conjugated antibody and developed with TMB. The array and detection chamber 1200 were then photographed as shown in FIG. 12B. Shown in the photographs are positive signals for Aldolase (left upper, “Aldolase 80 ng”), HRP2 (left lower, “HRP2 80 ng”), where the corresponding rows are positive for chromogen. Also shown is the combination of both antigens (right middle, “Both 80 ng each”), where all rows are positive by ELISA.

### 4. Assay Targets

Diagnostic detection of various pathogenic conditions and etiological agents of infectious diseases is contemplated, typically with card devices in the form of assay panels comprising both immunoassay and nucleic acid assay subcircuits. Blood-borne disease agents include Salmonella typhosa, Salmonella paratyphi, Bacillus anthracis, Brucella abortus, Brucella suis, Brucella melitensis, Yersinia (Pasteurella) pestis, Pasteurella multocida, Francisella tularensis, Spirillum minus, Burkholderia mallei, Leptospirum ictohaemorrhagiae, Coxiella burnetii, Rickettsia typhi, Hantavirus, Dengue fever virus, Yellow fever virus (and other viruses of the Flavivirus group), West nile virus, Japanese B encephalitis virus, St Louis encephalitis, Western equine encephalitis (and other viruses of the Arbovirus group), Human immunodeficiency virus 1 and 2, Human T-cell leukemia virus 1 and 2, Dirofilaria immitis in dogs, Plasmodium vivax, falciparum, malaria, ovale and berghei, to name a few. Differentiation of Plasmodium faliciparum from other febrile illnesses is of particular interest.

Wound and bite pathogens include Staphylococcus aureus, Streptococcus pyogenes serotypes responsible for necrotizing fasciitis, Pseudomonas aeruginosa, Clostridium perfringens, Clostridium tetani, Yersinia pestis, Bacillus anthracis, Bacteroides fragilis and Rickettsia species.

Central nervous system and CSF pathogens include Neisseria meningitides, Streptococcus pneumoniae, Listeria monocytogenes, syphilis, Haemophilus influenza serotype B, Acinetobacter spp, Escherichia coli, Enterobacter spp, Pseudomonas aeruginosa, Staphylococcus aureus, viral encephalides such as Japanese B encephalitis, Mumps virus, Polio virus, herpes viruses (HSV-1, HSV-2), varicella zoster virus, and Rabies virus.

Representative urinary pathogens are dominated by gram negative rods, and include Proteus mirabilis, Proteus vulgaris, Escherichia coli, Enterobacter cloacae, and occasional Pseudomonas aeruginosa infections, for example.

A panel for sexually transmitted diseases is contemplated. Pathogens of clinical interest include Neisseria gonorrhoea, Treponema pallidum, Herpes simplex, Chlamydia trachomatis, Papilloma virus, Candida albicans, Ureoplasma ureolyticum, Mycoplasma genitalia, and the like.

Enteric pathogens include Vibrio cholera and Enterobacteriaceae of the genera Salmonella, Shigella and certain serovars of E. coli, among others. Also pathogenic under the right circumstances are a broad swath of intestinal parasites and viruses.

Respiratory panels can include Streptococcus pyogenes, b-hemolytic Streptococci, Hemophilus influenza, Bordatella pertussis, Streptococcus pneumoniae, Klebsiella pneumoniae, Legionella pneumoniae, Corynebacterium diptheriae, Coxiella burnetti, Staphylococcus aureus, Mycoplasma sp, Pneumocystis cameii, Pseudomonas aeruginosa, Influenza viruses, type A and B, Parainfluenza viruses 1, 2, and 3, Adenovirus, Respiratory syncytial virus, Mycobacterium tuberculosis, Neisseria meningidites, Cytomegalovirus, Rhinovirus, as would be useful to screen for pandemic flu or to identify an etiological agent for non-specific or community acquired respiratory syndrome.

Panels may also be grouped by their clinical presentation. For example an Acute Fever panel could consist of Malaria, Measles, Dengue, Rickettsia, Salmonella, and Influenza. A more complete panel of fever agents would include the above, and Bartonella, Arbovirus, Corynebacterium diptheriae, Viral hemorrhagic syndrome agents, Leptospira, Pseudomonas pseudomallei, Meningoencephalitis agents, Bordatella pertussis, Yersinia pestis, Legionella, Chlamydia psittaci, Coxiella bumetti, Borellia, Rickettsia, Trichinella, Typhoid and paratyphoid organisms, and also fevers of unknown origin. Chronic fever panels would include added parasites, viruses and fungi. Recurrent fevers would include malaria, HIV and Borrelia, and so forth. Selected pathogens may be detected individually or in panels by the devices of the invention. Kits for detection of selected pathogens or pathogenic conditions are anticipated. Detection of gram positive cocci, gram positive rods, yeasts, and endospores, may require sample pretreatment in a mini-bead impact mill, ultrasound, or by peptidoglycanase or chitinase to lyse cells and spores prior to analysis.

### 5. Embodiments by Class

In addition to the microfluidic card devices described above, the invention encompasses methods for use of the cards in differential laboratory diagnostic procedures. The invention also encompasses an apparatus comprising the card device and a host instrument for operation of the card device. The invention also encompasses an algorithm for differential laboratory diagnosis based on test results from card devices of the present invention.

The methods are illustrated in the examples below, but more generally comprise comprising the steps of 1) Providing a microfluidic card (60, 70, 80, 90) to a user, the microfluidic card having at least one sample port (601, 700, 801, 820), the sample port further comprising a first valved fluidic connection (615) to a first microfluidic assay subcircuit and a second valved fluidic connection (616) to a second microfluidic assay subcircuit; wherein said first microfluidic assay subcircuit (20,71,81,90,91,92) is configured for performing a plurality of immunoassays, and said second microfluidic assay subcircuit (30,40,60,72,82) is configured for performing a plurality of nucleic acid assays; 2) Then introducing at least one biological sample collected from a single vertebrate host into the first sample port; 3) Interacting (i.e., docking) the microfluidic card with the host instrument, the host instrument having means for valvedly controlling both microfluidic assay subcircuits by commands from the user, and performing a plurality of assays on said microfluidic card; 4) Then reading a plurality of test results from said plurality of assays; 5) Making a differential laboratory diagnostic finding based on the plurality of test results; and 6) Discarding the microfluidic card in which is entombed the biological sample. In this way, the user collects a mixed panel of test results that can be correlated to arrive at a differential diagnosis.

Each assay subcircuit typically contains a panel of tests directed a particular diagnostic problem, such as determining the cause of a fever, or the presence of a sexually transmitted disease, or ruling out multiple co-infections, or determining the stage of a disease in a patient, and so forth. The assays can be run in parallel or in series under command of the user. The user can also select one of the assays and not run the other, because fluidic access to the assay subcircuits is controlled by valves. While blood is a preferred specimen, methods for running other samples, including swabs, can be adapted to the card devices. The host instrument typically will contain multiple subprograms that permit the user to run various assays and different combinations.

In another embodiment, the invention is an apparatus for performing differential laboratory diagnostic testing, and comprises:

a) a disposable, single-entry microfluidic card (60, 70, 80, 90) with plastic body, and a host instrument,

b) the microfluidic card having a first sample port (601, 700, 801, 820), a first microfluidic assay subcircuit (20, 71, 81, 90, 91, 92), which is configured for performing a plurality of immunoassays, and a second microfluidic assay subcircuit (30,40,60,72,82), which is configured for performing a plurality of nucleic acid assays. The sample port is configured for accepting a biological sample, and the sample port further comprises a valved fluidic connection (615) to the first microfluidic assay subcircuit;

c) The first microfluidic assay subcircuit comprises all reagents for performing said plurality of immunoassays and said second microfluidic assay subcircuit comprises all reagents for performing said plurality of nucleic acid assays; and,

d) The host instrument comprises a dock for receiving said microfluidic card and a microprocessor configured for valvedly controlling said first and second microfluidic assay subcircuits.

The apparatus requires a disposable card which contains the microfluidics and reagents for the assay, and also requires a host instrument to control the microfluidics (valves and pumps are used to direct fluid flow), to control temperature on the card in selected areas, and optoelectronics or other detection means to detect signals characteristic of test result endpoints. The host instrument is provided with a dock and docking interface for receiving and mating with the disposable card and with a microprocessor and logic instructions for performing a variety of assays once the card is in place. The host instrument is provided with a user interface for entering assay commands and optionally for entering patient data and for communicating with a remote network. Detailed instructions for the assays, the valve and fluid logic and step sequence, are generally pre-programmed and stored in non-volatile memory. The instruction set required may vary depending on the assay and the user may select assays according to the need at the time. The host instrument determines whether the program selected is compatible with the card inserted in the docking port. The host instrument then automatically controls the fluid logic needed to carry out the sequential fluid transfers and steps of the assay methods. The host instrument also supplies electrical power to the card if required, and is also supplied with a pneumatic control interface and contains a source of pressurized air, the utility of which is illustrated FIGS. 1-9 here. The host instrument can include thermal interfaces for heating and cooling selected zones or chambers in the card, such as Peltier chips or external resistive heating elements. These thermal interfaces can include conductive transfer surfaces, leads to embedded resistance heating elements on-card, or radiative heating devices. The host instrument can also include a magnetic interface for manipulating beads in the cards. The apparatus thus performs the steps of the method in a way that relies on interdependent properties of the card and the host instrument, such that the two do not have independent function. Manually operated cards can be designed, but have not generally been of interest. The cards are self-contained in that all reagents are supplied in the card, either as dried reagents printed or deposited in the microfluidics or as fluid reagents stored on-board in sachets or pouches which can be ruptured when the liquid is dispensed to the assay. However, the cards are not self-directing, and must be docked with the host instrument to perform the assay. In the assays described here, the host instrument operates valves, air ports, and bellows pumps where indicated in the schematics of the devices. A network of pneumatic control channels on the card interfaces with a control manifold on the host instrument. The control manifold consists of a few or many pneumatic nipples that interface with the card. Actuation of pressure in a pneumatic channel on command of a microprocessor controlling a solenoid results in opening or closing a valve on the card, or pumps a liquid, or ruptures a “blister pouch”, as required. The microprocessor operates with RAM or EPROM instructions and is clocked so that assay functions are actuated in the proper order and at the proper intervals.

The microfluidic card has been innovated to meet the needs of a new class of differential laboratory diagnostics by higher levels of integration of microfluidic circuits, combining multiplex immunoassay and multiplex nucleic acid assay capabilities in a single disposable device. In one embodiment the invention comprises a disposable plastic card body with immunoassay fluidic subcircuit; nucleic acid assay fluidic subcircuit; on-card waste sequestration chamber; and with sample port, the plastic card body further comprising all reagents for said differential laboratory diagnostic testing method.

In another embodiment, the microfluidic card for differential laboratory diagnostic testing comprises a) a single-entry, disposable card (60,70,80,90) with plastic body with sample port (601,700,801,820) for receiving a biological sample, said plastic card body further comprising all reagents for said differential laboratory diagnostic testing; b) An immunoassay fluidic subcircuit with first (206,710,810,900,904) and second bellows pumps (208,712,812,902,906), said sample port in fluidic connection to said first bellows pump, and at least one immunobinding test pad (212,713,813,906,907,908,909,910) interposed between said first and second bellows pumps; c) A nucleic acid assay fluidic subcircuit (30,40,60,72,82) with nucleic acid extraction subcircuit and with first (308,431,620,751,840) and second bellows pumps (309,432,621,752,841) said nucleic acid assay fluidic subcircuit in fluidic connection to said sample port; d) A detection chamber (331,451,642,761,870) in fluidic contact with said second bellows pump (309,432,621,752,841), said detection chamber further comprising at least one optical window for reading a plurality of test results; and, e) an on-board waste sequestration receptacle for entombedly disposing of said biological sample.

Immunoassays for a plurality of test targets can be multiplexed or simplexed with branching parallel assay channels and/or detection channels. Nucleic acid assay panels for a plurality of targets can be multiplexed or simplexed with multiple parallel amplification channels, and can include cDNA synthesis, and nested, symmetric and asymmetric amplification. Detection can also be multiplexed or simplexed with branching parallel assay channels and/or detection channels. Detection means include hybridization on arrays, lateral flow strips, FRET with or without temperature melt curve, and Magnaflow magnetic bead endpoint detection. The test results can be read visually in some cases and by optoelectronic devices in the host instrument in other cases.

The sanitary use of an on-board waste sequestration receptacle ensures that biological hazard placed in the card is entombed with the card on disposal. Other sanitary features, such as on-board reagents, diaphragms covering pump interfaces, closures on the sample inlet port, and hydrophobic filter membranes on vents, transform the microfluidic device into a product that can be safely handled and used. These self-contained cards are then packaged in kits for single-use diagnostic applications.

In some cards, two samples from a patient can be analyzed simultaneously, such as a blood sample and a swab, or blood and saliva, or two blood samples, as desired by the user. The immunoassay circuit is typically designed to process plasma or serum, but can be adapted to process saliva, urine, or other bodily fluids containing antibodies. The nucleic acid assay subcircuit has also been tested with blood-based samples, but the diagnostic power of PCR is exhibited in successful amplification of targets in a wide variety of bodily fluids and solid specimens, such as those collected by swab. It should now be apparent that analysis of a throat swab, for example, for an infectious agent, with simultaneous analysis of blood from that same patient for antibodies or antigens, is a more reliable means of screening for multiple pathogens with different patterns of virulence and routes of dissemination. The public health function is enhanced by this improvement.

Controllable valved fluidic interconnections between said immunoassay fluidic subcircuit and said nucleic acid assay fluidic subcircuit, whereby a user may select a plurality of immunoassays, a plurality of nucleic acid assays, or a combination of assays thereof, permits the user to tailor the panel to their clinical needs.

Combined assay cassettes for multifactorial laboratory diagnosis are innovative. Mixed testing can comprises differential serology, such as determination of antibody by class and by specificity. Mixed format assay kits include testing for multiple pathologies in one card device. Mixed format assay kits also include highly integrated multiplex or multiple, parallel, simplex nucleic acid detection assays for panels of targets. Various configurations are illustrated in the following examples.

### EXAMPLES

### Example 1

### ELISA Device

A first-order immunoassay card device was designed and manufactured for indirect ELISA assays of fluidized biosamples. The device features on-board sample processing, on-board reagents, a visual detection system, and sanitary design in a disposable, self-contained, single-entry, single-use package or kit. The device comprises fluidic subcircuits composed of microfluidic channels, vias, valves, reagent chambers with dehydrated reagents, mixing channels and chambers, blister pouches for liquid reagents, vents, pumps, all operated by a host controller with remote microprocessor linked by a manifold to the control surfaces of a pneumatic manifold integrated into the device, and in which the device is docked during operation. The combination of the device and the host instrument is an assay apparatus. Incorporation of multiplex or parallel multiple first-order devices into second-order integrated fluid handling systems achieves hithertofor unavailable holistic depth in differential laboratory diagnostics on a single card.

### Example 2

### TM-FRET Device

A first-order nucleic acid assay card device was designed and manufactured for nucleic acid PCR assays of fluidized biosamples. The device features on-board sample processing, on-board reagents, thermal interfaces, a FRET probe fluorescence detection system, and sanitary design in a disposable, self-contained, single-entry, single-use package or kit. The device comprises fluidic subcircuits composed of microfluidic channels, vias, valves, reagent chambers with dehydrated reagents, mixing channels and chambers, blister pouches for liquid reagents, vents, pumps, and parallel simplex detection chambers, all operated by a host controller with remote microprocessor linked to the control surfaces of a pneumatic manifold integrated into the device, and in which the device is docked during operation. The device further comprises microfluidic subcircuitry for sample processing and nucleic acid extraction, subcircuitry for target nucleic acid amplification, and subcircuitry for detection and reporting of assay data. The combination of the device and the host controller is an assay apparatus. Incorporation of multiplex or parallel multiple first-order devices into second-order integrated fluid handling systems achieves hithertofor unavailable holistic depth in differential laboratory diagnostics on a single card.

### Example 3

### Two-Tailed Amplicon Detection Device

A card device was designed and manufactured for nucleic acid PCR assays of fluidized biosamples. The device features on-board sample processing, on-board reagents, thermal interfaces, a magnetic interface, a two-tailed amplicon detection system with magnetic beads, and sanitary design in a disposable, self-contained, single-entry, single-use package or kit. The device comprises fluidic subcircuits composed of microfluidic channels, vias, valves, reagent chambers with dehydrated reagents, mixing channels and chambers, blister pouches for liquid reagents, vents, pumps, and parallel simplex detection chambers, all operated by host controller with remote microprocessor linked to the control surfaces of a pneumatic manifold integrated into the device, and in which the device is docked during operation. The device further comprises microfluidic subcircuitry for sample processing and nucleic acid extraction, subcircuitry for target nucleic acid amplification, and subcircuitry for detection and reporting of assay data, preferably in a visual format. The combination of the device and the host instrument is an assay apparatus.

### Example 4

### Integrated Nucleic Acid and Immunoglobin Diagnostic Device and Method for Plasmodium vivax

### Immunology

Infection with malaria results in an immune response. However, care is required in the selection of suitable recombinant antigens for indirect ELISA. Suitable antigens are often identified by the study of natural immune responses to human infections in the field. Plasmodium vivax merozoite surface protein 1 is thought to bind merozoites to the Duffy blood group antigen of reticulocytes and elicits an ELISA-competent immune response in natural infections.

### Molecular Biology

Great care is required in the selection of suitable primer pairs for detection of parasitemia by PCR. Using well-accepted rules for primer design, forward and reverse primers to genomic malarial DNA are designed from the GENBANK malarial database and compared with those used by other investigators. Primer pairs TCTCGTCAGCTGACGATCTCTAGTGC and ACGAGTGGGCCCTCCATCACATTTTTCTTT have been used in PCR of P. vivax-derived cDNA to amplify a genomic reticulocyte binding complex protein sequence of P. vivax merozoites. Microsatellite PCR of P vivax has also been described, with success in the use, for example, of forward primer CAAAGCCTCCAAATGAGGA and AT-rich reverse primer TTTTTGGCTTCTCACTCTGG, the primers having a melt temperature of about 55° C.

### Device Manufacture

A combination of immunoassay and nucleic acid assay subcircuits is built on a single card from stencils and laser cut laminates with dimensions selected to optimize fluidic performance. The card comprises the immunoassay subcircuit (20) of FIG. 2 and the nucleic acid assay subcircuit (40) of FIG. 4. A sample port and branched microfluidic channel with branching tee serves to split the sample into the two parallel assay pathways, and a common waste chamber on-card is also shared. The device as built contains all on-board reagents for the complete immunoassay and nucleic acid assay protocols. The device is also fabricated with a pneumatic manifold and interface for docking with a host controller, the host instrument serving as a source of pressurized air for the pneumatic manifold, for localized heating elements contacting the device, and is also supplied with a magnetic interface for the MagnaFlow assay (see below).

Reagents for a “sandwich” ELISA assay are prepared and packaged on the device. These include an enzyme-linked antibody and a chromogenic substrate for the enzyme in the dry state. Rehydration and Wash Buffer is packaged in co-laminated foil-backed plastic pouches, so-called “blister pouches”, in reagent chambers designed so that pressure on a deformable diaphragm apposing the pouch ruptures the pouch and releases of the contents under control of valve trees directed by the microprocessor and timed by the logic board clock of the host. The deformable diaphragm also serves to isolate the user from the reactants. The fluid in these blister pouches, once ruptured, can be dispensed in controlled increments under control of pressure on the diaphragms, which ranges from 1/100^{th} to 1/1000^{th} psig, and flow can be reversed by applying negative pressure of the same magnitudes. Thus, reciprocating flow regimes can be established to ensure dissolution of dried reagents prior to dispensation into the assay sample reaction mixture.

Purified pooled antibodies to P. vivax antigens are immobilized on plasma-treated polystyrene test pads in the detection chamber of a device of FIG. 2. Molecular biological reagents, where possible, are also dehydrated for storage. Many polymerases, nucleotides, and oligomers are reasonably stable when dried in a buffer salt matrix, although trehalose, dextrans, polyoxyethylene glycols, poloxamers, polyvinylpyrrolidinones, albumin, and other protectants have been useful and aid in rehydrating salt crystals and bioactive proteins. Primers, polymerase and other biologics are generally spot printed inside the reaction chambers during manufacture. Contact with the sample or with rehydration buffers dissolves them. Final concentrations are carefully optimized during pre-manufacturing validation.

The primers of this example are chemically modified at the 5′ end, for example as biotinylated primers or by conjugation with a hapten. Haptens are chosen for their molecular weight and for the availability of well characterized antisera. Protein and nucleic acid haptens may be used. Fluorogenic blunt-ended primers may also be used as haptens if suitable antisera are available. Selected quantities of primers are dried in place to support the PCR reaction.

Each test pad in the Magnaflow Detection Chamber is printed with a single immobilized antibody to one of the haptens used in the tagging of the individual primers. Thus only those tagged primers bearing that particular hapten will be recognized and immobilized on the test pad during the assay. Magnetic beads, avidin coated, are deposited in the Mag Bead Reservoirs, essentially as illustrated in the nucleic acid assay subcircuit of FIG. 4.

### Device Operation

Microscopy has its limits, and for greater sensitivity and fewer false negatives during the latent stages between fever spikes, an integrated microfluidic card of FIG. 4 combining subcircuits 20 and 40 is designed and manufactured. Anticoagulated whole blood is the sample of interest in the present example. Citrated whole blood, 50 uL, is introduced into the Sample Port of the card of this example. The device is then placed in the host instrument. Under microprocessor control, the blood sample is split by aspiration at a “tee” in the microfluidic channels on the device. About 30 uL is directed onto a polypropylene depth filter (Cell Separation Filter) for plasma separation. Plasma is collected in the Plasma Filtrate Chamber. About 20 uL is directed into a Lysis Chamber, and is treated with a chaotrope such as a weakly acidic guanidinium salt/detergent lysis buffer from the Lysis Buffer Pouch, which inactivates nucleases and disrupts nucleic acid:protein associations. The operation of the nucleic acid assay subcircuit can be followed on FIG. 4.

Beginning from the plasma filtrate, with reference to FIG. 2, immunoreactive antigens are captured by binding to the immobilized hybridoma or hyperimmune antibody on the test pads in a Detection Chamber of the ELISA fluidic subcircuit on the device. Bellows pumps at each end of the Detection Chamber cause the sample to flow back and forth across the test pads to maximize interaction and binding. When Wash Buffer is introduced into the chamber, the paired bellows pumps assist in rinsing the test pads and discarding the rinse to waste. This process is repeated as required. A measured volume of Wash buffer is then used to rehydrate the enzyme-linked anti-antigen reagent and dispense it into the detection chamber. ELISA antibodies against parasite antigens are dissolved in the reagent chamber and incubated with the test pads for up to 30 min, optionally at 37° C., in an optimized buffer. The test pads are rinsed again thoroughly before chromogenic enzyme substrate is added from a second reagent chamber when dissolved by Wash and Rehydration Buffer. The addition of antibody and chromogen are controlled by separate valves.

Chromogen typically precipitates on the test pads where sandwich antibodies bound to enzyme have been captured on the immobilized antigen:target antibody complexes, a principle well known to those familiar with indirect ELISA assays. Typical enzyme:chromogen systems are known in the art, and include for example the horseradish peroxidase and TMB (tetramethylbenzidine) pair, which is used here. Reduced TMB forms a bright blue lake in the test pad. The assay endpoint is read through an optical window. Positive immunoassay data is useful in detecting an active infection. Similarly, detection of antibodies and identification of antibody class promote a more complete picture of the infection.

The whole blood lysate or cellular fraction lysate, optionally supplemented with plasma discarded from the ELISA fluidic subcircuits (note the valved return channel 717-718 from the ELISA reaction back to the nucleic acid capture chamber of FIG. 7), is introduced into the Nucleic Acid Target Capture Assembly and nucleic acids are captured on a glass fiber filter or similar solid phase bed material. Here silica fiber is used. After washing the retentate with a Solvent Wash solution, often alcoholic, and drying under blowing air, the nucleic acid retentate is eluted with Elution Buffer, a low salt, slightly basic buffer optimized for elution and PCR, and the buffer, carrying solubilized nucleic acids, is directed into the PCR fluidics subcircuitry (or other nucleic acid amplification means), where the next phase of the assay takes place.

PCR is performed in a fluidic subcircuit 40 (FIG. 4) equipped with a thermal interface, here shown as having two distinct temperature stations and two bellows chambers, the first most set a temperature to melt double stranded DNA species, the second at a lower temperature selected to promote annealing and primer extension. Forward and reverse primers are generally printed in place during manufacture and dehydrated in the vestibule of the first amplification chamber or chambers, as is dried polymerase, an optimized mass of magnesium salt, and sufficient dNTPs to sustain the reaction through multiple thermocycles. The dehydrated reagents are rapidly dissolved and mixed upon heating the elution buffer reaction mixture. The number, time, and temperature targets of the thermocycling protocol are optimized as is customarily practiced by those skilled in the art. A negative control channel is also shown, and assists in identifying problems with contamination of the device during manufacture or handling.

In the current example, which detects a visual endpoint, about 40 thermocycles are performed in the PCR fluidics and thermocycling subassembly, which consists of paired bellows pumps, one pump held at denaturation temperature and the other at the annealing temperature of the primer pair. Reaction volume is about 50 uL. Typical results may be obtained with 30-45 cycles. Cycle time is about 30 sec, and temperatures of 96° C. for melt and 45° C. for anneal are chosen, as is dependent on the primers and the buffer or solvent matrix. A molar excess of forward and reverse primers are provided. These forward and reverse primers have been specially tagged, and become incorporated in the amplicon products of PCR. Simplex amplification is shown here.

Following amplification, the reactant mixture is mixed with avidin-coated magnetic beads that have been rehydrated by a Rehydration and Wash Buffer. The mixing occurs in a Mixing Chamber and can be augmented by the reciprocal pumping action of the Mag Mixer and Mag Bead Reservoir Chambers. When homogeneous, the mixture is opened up into the Detection Chamber and is subjected to a magnetic field that propels the beads back and forth the chamber, bringing them into close contact with the test pads. The test pads are then rinsed with buffer from the Rehydration and Wash Buffer pouch, and positive nucleic acid assays are recorded for those test pads that are colored with a bright rust colored pigment characteristic of bound magnetic beads. Note that multiple targets can be detected in a single detection chamber by providing multiplex, discreet test pads, each with a particular antibody. Detection in this kind of detector is most often multiplex, as shown in FIG. 4. Clear visual signals are obtained for positive results.

Put in other words, avidin coated magnetic beads are used to capture biotin-labeled amplicons. Any two-tailed amplicons also containing the hapten-tagged second primer, are then also captured on test pads coated with specific antibody to the hapten. In this way, test pads that become colored due to capture of the magnetic bead:two tailed amplicon complexes are consistent with the diagnosis of P. vivax. While the endpoint is visual, it can be machine read optoelectronically or read by the user. This second order disposable card provides a hithertofor unavailable holistic view of the patient's condition, with improved sensitivity and specificity.

### Example 5

### Integrated Nucleic Acid and Immunoglobin Diagnostic Device and Method for P. vivax. Use of FRET Probes (“Molecular Beacons”)

For this example, the device of FIG. 7 is the disposable card used in the analysis. The host instrument is adapted to accommodate a variety of microfluidic disposable card devices and contains programmed instructions for various assays. Here P. vivax is again the target. Antigens, antibodies and blood nucleic acids are detectable in an active infection, although the nature of any mRNA nucleic acid target may vary with the tertian cycle of the fevers.

Primers, polymerase and other biologics for PCR are generally spot printed inside the reaction chambers during manufacture. Contact with the sample or with rehydration buffers dissolves them. The endpoint for nucleic acid target detection here uses a adaptation of FRET probe technology. FRET probe chemistries (also termed “molecular beacons”) are familiar to those skilled in the art. These probes may be designed to light up when hybridized (or when denatured) and their melt characteristics can be predicted by calculations similar to those used in designing primers.

FRET probes are spotted in the PCR amplification subcircuitry, so that the signal of the annealed probe is immediately evident when the nucleic acid amplification reaction product enters the detection chamber and is illuminated by epifluorescence by host instrument optics. The detection chamber interfaces with a variable temperature thermal interface on the host controller. Using the device, a melt curve of the fluorescence signal can be acquired, and a first derivative plotted with off-device data analysis software.

### Device Operation

Citrated whole blood, 50 uL, is introduced into the sample port of the device of this example. The disposable card device is then docked in the host instrument controller. Under microprocessor control, the blood sample is split by aspiration at a valved “tee” (705, 706) in the branched microfluidic channels on the device as shown in FIG. 7. About 30 uL is directed onto a polypropylene depth filter (701, cell separation filter) for plasma separation. Plasma is collected in the plasma filtrate chamber (702). About 20 uL is directed into a lysis chamber (730), and is treated with a chaotrope such as a weakly acidic guanidinium salt/detergent lysis buffer from the Lysis Buffer Pouch, which inactivates nucleases and disrupts nucleic acid:protein associations. The operation of the device can be followed on the FIG. 7 schematic.

The whole blood lysate or cellular fraction lysate, optionally supplemented with plasma discarded from the ELISA fluidic subcircuits (note the valved return channel 717-718 from the ELISA reaction back to the nucleic acid capture chamber of FIG. 7), is introduced into the Nucleic Acid Target Capture Assembly 731 and nucleic acids are captured on a glass fiber filter or similar solid phase bed material. Here a silica fiber matt is used. After washing the retentate with a Solvent Wash solution, often alcoholic, and drying under blowing air, the nucleic acid retentate is eluted with Elution Buffer, a low salt, slightly basic buffer optimized for elution and PCR, and the buffer, carrying solubilized nucleic acids, is directed into the cDNA sSynthesis chamber 740 and treated with reverse transcriptase at a controlled temperature. Reverse transcriptase can be inactivated by heating to >70° C. for several minutes. Following incubation, the cDNA product is directed to the PCR fluidics subcircuitry 750, where the next phase of the assay takes place, generally after dilution and reconstitution of the matrix, including supplemental magnesium salt, polymerase and cofactors.

PCR is performed in a fluidic subcircuit equipped with a thermal interface, here shown as having two distinct temperature stations (758,759) and two bellows chambers (751,752), the leftmost set a temperature to melt double stranded DNA species, the second at a lower temperature selected to promote annealing and primer extension. Forward and reverse primers are generally “printed” in place during manufacture and dehydrated in the vestibule of the first amplification chamber or chambers, as is dried TAQ polymerase, an optimized mass of magnesium salt, and sufficient dNTPs to sustain the reaction through multiple thermocycles. The dehydrated reagents are rapidly dissolved and mixed upon heating the elution buffer reaction mixture. In the current example, after the extracted nucleic acid enters the PCR fluidics subcircuit, 45 thermocycles are performed. Cycle time is about 30 sec, and temperatures of 96° C. for melt and 45° C. for anneal are chosen, as is dependent on the primers. A molar excess of forward and reverse primers are provided.

Following amplification, the reactant mixture is pumped into a detection chamber (here, TM-FRET detection chamber and optical window 761) The detection chamber of the present example can contain a dehydrated molecular beacon specific for one target cDNA of interest, or more commonly, the single-stranded molecular beacon probe is contained in the PCR reaction mixture added earlier, thus eliminating the need for a final denaturation and re-annealing. In other embodiments, up to 4 such molecular beacons (each having a distinct fluorophore) may be provided in a single detection chamber or reaction mixture, and alternatively, multiple detection chambers in parallel may be provided.

This embodiment of the invention uses a detection chamber assembled with a thermal interface 763 so that a thermal melting curve of the double stranded amplicons in the presence of a FRET probe can be performed. Temperature in the chamber is ramped while monitoring the fluorescent signal of the molecular beacon. This provides two data, the first confirming the presence of immunological binding between amplified target and the FRET probe, the second confirming the specificity of the binding by the detection of the expected melt curve. The optoelectronic package for monitoring fluorescence is provided in the host instrument. An optical window in the card device facilitates this measurement.

In these second-order integrated devices, the combined information from the molecular biological testing and immunoassay, taken together, strengthen the diagnostic power of the device, and provide important clinical information about the stage and progress of the infection. Typically, patients presenting with malaria-like recurrent or chronic fever will be infected with one or more strains of malaria, or possibly with another etiological agent such as Borrelia or HIV. A panel combining immunoassay and nucleic acid assay panel testing reduces the uncertainty of the differential diagnosis and adds sensitivity and selectivity.

### Example 6

### Data from a Thermal Melt Curve of Target DNA in the Presence of a FRET Probe

Representative data for positive test results with amplicon and molecular beacons as probes are shown in FIGS. 11A and 11B, which was obtained with clinical specimens. A thermal melting curve and negative control are shown in FIGS. 11A and 11B. The FRET approach with temperature melt curve has the advantage of detecting false positives due to primer dimers because the temperature melt curve does not match. The FRET probes used generated a fluorescent signal upon hybridization with the target amplicon and are representative of molecular beacons in the public domain, trade secret molecular beacons, and patented molecular beacons. As the temperature was ramped from about 55 to 95 C, the hybridized probes are melted off and the signal is quenched. Shown are positive test results and melt curves for Malaria (FIG. 11A), and Salmonella (FIG. 11B).

### Example 7

### Integrated Nucleic Acid and Immunoglobin Diagnostic Device and Method for Plasmodium falciparium

### Biology

Malignant Plasmodium falciparum parasitemia is not associated with episodes of fever at regular intervals, rather the fever can be continuous or semi-continuous due to near continuous release of merozoites into the bloodstream from sporozoites sequestered in the liver. Also called “blackwater fever”, the parasitic load on the body can become so large as to result in kidney failure due to the excess of hemoglobin released from bursting red cells. P. falciparum is also unique in causing cerebral malaria and severe anemia. Ring forms of P. falciparum in blood may exceed 1000 per microliter or in extreme conditions, 10,000 per microliter. However, endemic P. falciparum malaria is also characterized by a high prevalence of chronic infections with very low, fluctuating, parasite densities; thus emphasizing the need for diagnostic tools to supplement or supplant thick smears.

### Immunology

Infection with Plasmodium falciparum is associated with appearance of antibodies in chronic and acute infections and in convalescent sera. ELISA-reactive antibodies to recombinant merozoite antigens, including AMA-1 (apical membrane antigen 1), MSP-1 (a 19-kDa C-terminal region of merozoite surface protein 1, antigenic variants of MSP-1 with double and triple substitutions (E-KNG, Q-KNG and E-TSR), and MSP-3 (merozoite surface protein 3), have been detected. Plasma antigens are also present, and include pan-specific aldolase and type-specific HRP2.

Schizonts are generally rare in circulating blood because of adhesion to capillary endothelia, a critical event in the pathology of P. falciparum malaria. CLAG-9 (Cytoadherence-linked asexual protein 9), which is in part responsible for this adhesion, elicits strong antibody responses in patients and interestingly, can also be detected by PCR during parasitemia. Antibodies to the P. falciparum karyopherin beta (PfKbeta) homologue localized in the parasitophorous vacuole at the schizont stage are also found in immune sera. EBP2/BAEBL (erythrocyte binding protein 2) and MAEBL also elicit strong antibody responses and interestingly, are detectable during parasitemia by reverse transcriptase-assisted PCR of infected erythrocyte lysates. Another blood stage antigen associated with significant antibody titers is SERA5, which is also detectable by reverse transcriptase assisted PCR in infected plasma.

### Molecular Biology

Using well-accepted rules for primer design, forward and reverse primers to schizont adhesion complex mRNA transcripts are designed from the GENBANK malarial database and checked against those used by other investigators. Trophozoite-related mRNAs may also be targeted.

### Device Manufacture

Recombinant purified pooled antigenic complexes of P falciparum schizont surface and adhesion proteins are immobilized on plasma-treated polystyrene test pads in the detection chamber of a device of FIG. 7. Serine repeat antigen SERA5 and erythrocyte binding protein-2 EBP2/BAEBL are chosen for this example. Reagents for a “sandwich” ELISA assay are prepared and packaged on the device in foil-backed plastic pouches in a reagent chamber designed so that pressure on a deformable diaphragm results in rupture of the pouch and release of the contents at the appropriate time in the assay protocol.

A chamber for reverse transcriptase is provided on the device. Included is a thermal interface for regulating the temperature for cDNA synthesis at about 37° C. Cofactors, including dNTPs and enzymes are provided in dehydrated form in the reverse transcriptase chamber.

The forward primers are chemically modified at the 5′ end as biotinylated primers and the reverse primers are conjugated with a hapten for which a well characterized antiserum is available. Dried primers, dNTPs, probes, and TAQ polymerase are spot printed inside the PCR fluidics subcircuitry on the device. Reverse transcriptase in a suitable buffer, with an antisense primer, dNTPs and cofactors, is deposited in the cDNA Synthesis Chamber, which has independent temperature control, typically at a fixed point between 30 and 55° C.

### Device Operation

EDTA whole blood, 50 uL, is introduced into the whole blood sample port of this device. The disposable card is then placed in the host instrument, which controls valves and pumps pneumatically, provides the needed heating interfaces, and can include an optoelectronic package for reading assay results. A polypropylene depth filter (Cell Separation and Lysis) is used to separate plasma from the cellular fraction of blood. The cellular retentate on the filter is treated with Lysis Buffer, a chaotrope such as a weakly acidic guanidinium salt/detergent and the lysate is rinsed into the Lysis Pool, where it is joined by plasma returning from the ELISA assay.

From the Plasma Filtrate, immunoreactive IgG antibodies against the malarial antigens are then captured on the antigen test pads in the Detection Chamber of the ELISA fluidic subcircuit on the device. ELISA is continued by washing of the test pads with diluent from the Wash Buffer Pouch. The detection chamber is then flooded with reconstituted immobilized antibodies, ELISA antibodies against human immunoglobin classes IgG 1-3, and incubated for up to 30 min, optionally at about 35° C., in an optimized buffer. The test pads are then rinsed thoroughly with Wash Buffer to remove unbound antibody, before Chromogenic Enzyme Substrate is added. The substrate typically precipitates as a colored lake on the test pads where sandwich antibodies have been captured on the immobilized antigen:target antibody complexes, a principle well known to those familiar with ELISA assays. Typical enzyme:chromogen systems are known in the art, and include for example the horseradish peroxidase and TMB (tetramethylbenzidine) pair, which is used here.

From the cell lysate, nucleic acids are captured on a silica fiber filter or similar solid phase bed material of the Nucleic Acid Target Capture Assembly. After washing with an alcoholic solution, and drying under blowing air, the nucleic acid retentate, including mRNAs, is eluted with elution buffer and transferred to a cDNA Synthesis Chamber, where first strand cDNA copies of the mRNA species in the lysate are made. Any one of several reverse transcriptases may be used. Incubation is at a fixed temperature, generally 30-55° C. for 10 to 30 minutes. It is the product first strand cDNA copies, plus any parasite-derived genomic DNA, that are the target of PCR amplification in the next phase of the assay.

PCR is performed with FRET detection. FRET probes may be public domain, proprietary or patented, and are known to those skilled in the art without further elaboration here. In the method of the present invention, it is the combined information provided from molecular biological testing and immunoassay that provides added assurance in the diagnosis and progress of the infection.

### Example 8

### Integrated Nucleic Acid and Immunoglobin Diagnostic Device And Method for Dengue Virus

### Immunology

Dengue Virus is the cause of “bone-break” fever, and produces an acute fever and long convalescence. There is clinical interest not only in identifying the Dengue Virus serotype in each infection, but also in determining the nature of the antibodies present (i.e., IgG versus IgM). Capture antibody sandwich serology is a frequent test performed in Dengue laboratory workups.

### Molecular Biology

Dengue is a single stranded “sense” RNA virus. Active infection results in large amounts of sense and antisense RNA in blood. The virus carries its own RNA replicase. Primers for molecular biological diagnostic assays are selected based on highly conserved regions of Dengue Virus genome. The complete genome sequence (of about 11 Kb) is known for representative strains of all four dengue virus serotypes.

Sequence alignment of the serovars is performed with DNA Star software (Perkin-Elmer) and potential target regions are identified in the core, NS3, NS5, and 3′ noncoding genes. The following primer pairs have been reported in the literature to be reliable and can be multiplexed when used at appropriate concentrations. Both group and serotype specific primer pairs are provided. A universal forward primer can be used. The shortest amplicon is 133 bp; the longest 203 bp.

| GenBank | | | |
|---|---|---|---|
| Ref | NT | | SENSE PRIMER |
| AF038403 | 135-158 | Dengue Group | CAATATGCTGAAACGCGAGA |
| | | | GAAAC |
| Genbank | | | |
| Ref | NT | | REVERSE PRIMER |
| AF038403 | 282-305 | Dengue Group | CCCCATCTATTCAGAATCCCT |
| | | | GC C |
| AF180817 | 304-325 | Serotype 1 | CGCTCCATACATCTTGAATGA |
| | | | GC |
| AF038403 | 319-338 | Serotype 2 | AAGACATTGATGGCTTTTGAC |
| M93130 | 316-336 | Serotype 3 | AAGACGTAAATAGCCCCCGA |
| | | | CC |
| M14931 | 245-268 | Serotype 4 | AGGACTCGCAAAAACGTGAT |
| | | | AATC |

The above primer pairs are specific for Dengue virus. A primer pair that picks up all flavivirus species generically is GCCATATGGTACATGTGG and TGTCCCATCCTGCGGTATCAT.

### Device Manufacture

During manufacture of the device, affinity purified goat anti-human IgG and anti-human IgM antibody (specific for Fc region) are immobilized with drying and heat on separate plasma-treated polystyrene test pads in the upper Detection Chamber 901 of the dual ELISA subcircuit of the device of FIG. 9. In the lower Detection Chamber 905, other tests pads are spotted with culture supernatant antigen derived from each of the serovars of Dengue virus. Including controls, a total of 9 test pads are masked out in the two Detection Chambers during manufacture. Test pads are well separated by inert surfaces during plasma treatment and spotting. After the masking material is removed, all test pads and the reaction vessel surfaces are then carefully treated with blocking agent.

Reagents and antibodies for a capture-type antibody-sandwich ELISA are prepared and packaged on the device. Liquid reagents are packaged on the device in foil-backed plastic pouches in reagent chambers designed so that pressure on a deformable diaphragm results in rupture of the pouch and release of the contents at the appropriate time in the assay protocol. Required are Wash Buffer (which is also used to reconstitute other dehydrated immunological reagents), pooled viral antigens (for the sandwich), mouse hybridoma IgG anti-viral antibody, enzyme-linked anti-mouse antibody, and chromogenic substrate for the ELISA.

Reagents and antibodies for an indirect “sandwich” ELISA assay are prepared and assembled in the device before completion of manufacture. Required are Wash buffer, enzyme-linked goat anti-human antibody, and chromogenic substrate for the ELISA.

A chamber for reverse transcription of RNA is provided on the device (cDNA Synthesis Chamber). Included is a thermal interface for regulating the temperature for cDNA synthesis at about 45° C. Cofactors, including dNTPs and enzymes are provided in dehydrated form in the reverse transcriptase chamber. Dried primers, dNTPs and TAQ polymerase are spot printed inside the PCR fluidics subcircuitry on the device. Multiplex, simplex, nested, or asymmetric PCR may be used.

### Device Operation

Into the sample port, a 50 uL plasma or serum sample is introduced into a microfluidic device fabricated with parallel immunoassay and nucleic acid assay subcircuits. We first focus on a detail of the immunochemistry and the corresponding device as pictured in FIG. 9. After docking the microfluidic device in the host instrument, the sample is split fluidically at a “tee” between the upper Detection Chamber for sandwich-antibody capture and the lower Detection Chamber for serovar-specific indirect ELISA.

Dual bellows chambers permit reciprocal pumping of the reaction fluid across the test pads, augmenting the speed of the reactions. Incubations are typically for 10 min. Immunochemistry of the upper and lower chambers is different, but the results are synergic. In the upper chamber, the patient's antibodies are sorted by class, here into IgG and IgM. Using the hydraulic action of dual bellows elements, the serum is allowed to wet the test pads and incubated with mixing for 10 min. From a reagent pouch, the detection chamber is then flooded with diluted pooled virus antigens from culture supernatants of the four serovars. Mouse anti-dengue group antibody is then added and the incubation continued for 10 min. After 3× washings with small volumes of wash buffer, a 1:1000 diluted horseradish peroxidase-conjugate goat anti-mouse IgG is added. Then, substrate is added and the color allowed to develop. Blue precipitate on the antigen treated test pads indicates which of the serovars is involved in the infection. Blue precipitate on the capture test pads treated with goat anti-human IgG or IgM indicate the antibody class of the immune reaction, if any. The endpoint differentiates the antibody by class; when IgG is present, the anti-human IgG test pad will light up; if the antibody is IgM, the IgM test pad will light up. This information is clinically useful in differentiating fresh infections from convalescent or chronic ones. In the lower Detection Chamber, the antigens on the test pads are specific for a group antigen of Dengue, and for serovar specific variants representative of each of the 4 major subclasses of the virus. Antibodies in the serum bind to those antigens, and the antibodies can then be detected by standard ELISA techniques. A positive test indicates an early, late, or chronic infection.

For nucleic acid analysis, serum sample nucleic acids are first captured on a silica solid phase support following treatment of the serum with a chaotropic salt and detergent. The solid phase is washed with a solvent or solvent-water mixture, and finally the nucleic acid retentate is eluted with a low ionic strength buffer suitable for the subsequent reactions. RNAsin is not used. The molecular biological subcircuits will detect actual pathogens in the blood of the patient, and the use of reverse transcriptase improves the minimum copy number at the limit of sensitivity. Dengue virus is a sense RNA virus, and like other flaviviruses, must first synthesize an RNA Replicase before it can initiate an infection. Thus, the initial stage of diagnosis by PCR is typically the use of an antisense primer and reverse transcriptase to synthesize first-strand cDNA.

To make first strand cDNA from viral RNA, a reverse transcriptase reaction is run 30 min in a heated reaction chamber (45° C.) containing optimal concentrations of dehydrated magnesium salt, dNTPs, reverse transcriptase, serotype specific reverse primers, and optionally the dengue and flavivirus group reverse primers CCCCATCTATTCAGAATCCCTGCC and TGTCCCATCCTGCGGTATCAT respectively. The conditions and biochemistry of reverse transcriptase reactions are well known in the art, but must be adapted for use in microfluidic devices.

The reaction mixture is then transported fluidically to a PCR reaction chamber containing additional reagents, including TAQ polymerase, nucleotides, salts and at least one primer. Reverse transcriptase is typically inactivated with heating during the first denaturation cycle. Optionally, the reverse transcriptase products are also diluted, for example from a few microliters to a few tens of microliters, when transferred to the PCR chamber. This is accomplished by the addition of water from an on-board reagent pouch.

Using a dual constant or variable temperature thermal interface, PCR thermocycling is conducted with a melt temperature of 95 C and an annealing/extension temperature of 55 C. The device is programmed to perform 40 cycles at 45 sec/cycle. Positive and negative control channels are also on-board.

Following PCR, the reaction mixture is hydraulically pulled into a thermally controllable detection chamber containing up to four dehydrated molecular beacons with individual fluorophores. Following denaturation and reannealing of the FRET probes to the target amplicons, fluorescent signals of positive test results are detected through a quartz window. To simplify the protocol, molecular beacons may instead be deposited with the PCR reaction mixture and thus enter the detection chamber active and ready for temperature ramping. Referencing FIG. 6, individual “simplex” detection channels are provided for each molecular probe. The reaction mix is diluted and split for detection.

Melting curves on the individual amplicons can be performed by raising the temperature of the annealed strands and probes from 55 to 95° C. with a variable thermal interface. The combined information provided from molecular biological testing and immunoassay provide added assurance in the diagnosis and important clinical information about the progress of the infection. Similar protocols in this device may be used to detect various arboviruses for example, including West Nile Virus, Yellow Fever Virus and Japanese Encephalitis Virus, using known primers, antigens and antibodies. Highly integrated panel assays are conceived.

### Example 9

### Integrated Nucleic Acid and Immunoglobin Diagnostic Device And Method for Measles Virus

### Biology

Measles Virus is transmitted via the respiratory route and has an incubation phase of 9 to 19 days. Large amounts of antibody to nucleocapsid protein developed in all patients by day one of a rash characteristic of the end of the prodromal period of Measles. Antibody to hemagglutinin develops in all patients over the next 3 weeks.

Whereas the cellular immune response is thought to be crucial for clearance of infection, virus-neutralizing antibodies (VN), primarily to the hemagglutinin, may also be important in opsonizing the viral particles. Not surprisingly then, VN antibody titers of 1:8 or 1:16 or higher have been shown to protect from disease. Thus while immunoassays may be predictive of recovery, they may not be sufficiently sensitive for early diagnosis.

### Molecular Biology

Measles is a single stranded “antisense” RNA virus which contains a pre-formed RNA-dependent RNA replicase. The negative-stranded nonsegmented RNA genome of the Measles Virus encodes eight proteins, including the nucleocapsid and the hemagglutinin proteins. The nucleocapsid sequence is bounded on the 3′ terminus by a hypervariable region. Sequence diversity within the complete H gene and the hypervariable region of the N gene (nt 1233 to 1682) classifies MV strains into eight clades (A to H) with a total of 22 different genotypes (A, B1, B2, B3, C1, C2, D1, D2, D3, D4, D5, D6, D7, D8, D9, E, F, G1, G2, G3, H1 and H2). Most MV genotypes have a more or less characteristic geographic distribution.

As described in the literature, selection of primers for reverse transcriptase assisted PCR is accomplished by aligning the hypervariable region of the nucleocapsid genes using DNA Star and identifying target clade and genotypic sequences with a common, conserved 3′ terminal primer. A universal PCR reverse primer sequence is GGGTGTCCGTGTCTGAGCCTTG.

Clade-specific forward primers are identified in the literature. These primers have been selected for use at an annealing temperature of 66° C., a relatively stringent condition. Efficient primer elongation is dependent on a matching nucleotide at the 3′ end. A mismatch at this position can inhibit elongation of incorrect primer hybrids. But high GC content near the 3′ terminus of the primer can promote elongation at a mismatched 3′ base, such as with primer CIB above, and can contribute to the formation of primer dimers. Specificity is also increased by using short annealing and elongation times (10 s). No single optimal condition is possible for all primers. Primer lengths, concentrations, melt temperatures, and 3′ terminal GC content, are all factors in designing a multiplex PCR primer mix.

| Clade mix | NT | Sense Primer |
|---|---|---|
| CIA | 1299-1321 | GCAATGCATACTACTGAGGACAA |
| CIB | 1563-1583 | CAGGACAGTCGAAGGTCAGCC |
| CIC | 1396-1420 | CGAGATGGGGGGGTAAGGAAGATAT |
| CIDa | 1374-1397 | GATCAAAGTGAGAATGAGCTCCCA |
| CIDb | 1374-1397 | GATCAAAGTGAGAATGAGCTACCA |
| CIDc | 1374-1397 | GATCAAAGTGGGAGTGAGCTACCA |
| CIG | 1468-1486 | CCGGGCACAGCAGAGCAAA |
| CIH | 1529-1549 | CATTGACACTGCATCGGAGTA |

Genotype-specific primers are identified in the literature.

| Genotype B Mix | NT | Sense Primer |
|---|---|---|
| GrB3.1 | 1567-1586 | ACAGTCGAAGGTCAGCCGAT |
| GrB3.2 | 1414-1434 | AGGACAGGAGGGTCAAACAGG |
| Genotype | | |
| DI Mix | NT | Sense Primer |
| GeD2 | 1462-1482 | GAGAAACCGGGTCCAGCAGAA |
| GeD4 | 1341-1365 | CCCAGACAAGCCCAAGTGTCATTTA |
| GeD6 | 1523-1548 | CCTAGACATTGACACTGCATCGGAGA |
| GeD9 | 1425-1448 | GTCAAACAGAGTCGGGGAGAAGCA |
| MVN | 1599-1619 | CTGCAAGCCATGGCAGGAATC |
| Genotype | | |
| DII Mix | NT | Sense Primer |
| GeD3 | 1500-1520 | GCCCATCCTCCAACCAGCATG |
| GeD5 | 1260-1280 | GGTATCACTGCCGAGGATGCG |
| GeD7 | 1553-1575 | CCAAGATCTGCAGGACAGCCGAC |
| GeD8 | 1439-1458 | GGGAGAAGCCAGGGAGAGCA |
| MVN | 1599-1619 | CTGCAAGCCATGGCAGGAATC |
| Genotype G Mix | NT | Sense Primer |
| GeG2 | 1482-1503 | GCAAATGATGCGAGAGCTGCTG |
| GeG3 | 1396-1423 | CGGGATTGGGGGGTAAGGAAGATAA |
| | | GAA |
| Genotype H Mix | NT | Sense Primer |
| GeH1 | 1342-1365 | CCAGGCAAGCCCAAGTCTCATTTT |
| GeH2 | 1457-1477 | CTACAGAGAAACCGGGCTCAA |

The primers used in this example are chemically modified at the 5′ end. The reverse primer is biotinylated. Forward primers are conjugated individually with a hapten for which a well-characterized antiserum is available.

### Device Manufacture

During manufacture of the device, test pads in the ELISA subcircuit are spotted with recombinant hemagglutinin and nucleocapsid antigen derived from the serotypes of Measles Virus. Test pads are masked out in the detection chamber during manufacture. Test pads are well separated by inert surfaces during spotting. After the masking material is removed, all test pads and the reaction vessel surfaces are then passivated and carefully treated with blocking agent.

Reagents and antibodies for an indirect “sandwich” ELISA assay are prepared and packaged on the device in foil-backed plastic pouches in reagent chambers designed so that pressure on a deformable diaphragm results in rupture of the pouch and release of the contents at the appropriate time in the assay protocol.

A primer selected from the nucleocapsid open reading frame, antisense reverse primer TTATAACAATGATGGAGG (nt 1740-1722), dNTPs, suitable quantities of magnesium salt, and a reverse transcriptase, for example Moloney murine leukemia virus reverse transcriptase (Invitrogen, Merelbeke, Belgium), are deposited in dehydrated form in the reverse transcription chamber of the device. This chamber is fitted with a thermal interface for incubation at a steady 42° C. during the reverse transcriptase reaction. Nested PCR is then performed on products of this reaction.

PCR primers, dNTPs and Platinum TAQ polymerase (Invitrogen, Merelbeke, Belgium) are spot printed inside the PCR fluidics subcircuitry on the device. Magnetic beads, avidin coated, are deposited in the Mag Bead Reservoirs.

### Device Operation

Citrated whole blood, 100 uL, is introduced into the whole blood sample port of this device. The device is then mounted in the docking port of the host instrument. The entire volume is directed onto a polypropylene depth filter for plasma separation. The plasma sample is then split by aspiration into the ELISA and PCR subcircuits of the device.

About 25 uL of plasma is directed into a lysis chamber, and is treated with a chaotrope such as a weakly acidic guanidinium salt/detergent lysis buffer to open viral capsids and disperse the nucleic acid contents. About 25 uL of plasma enters the immunoassay subcircuit 90 of FIG. 9 and is split between subcircuits 91 and 92. In subcircuit 92, immunoreactive mouse hybridoma anti-antibodies specific for the Fc domain of IgM and IgG sub-types are immobilized on the test pads. After selective binding of immunoglobins by class, any unbound plasma proteins are then diverted to waste or recovered for mixing with the plasma in the lysis chamber. The bound antibody is then treated with measles antigen and with conjugated goat anti-measles and detected by ELISA. Immunoassay subcircuit 91 contains virus antigen divided among test pools by serovar. Host antibodies to particular serovars are detected by ELISA.

Following washing of the immobilized patient antibodies with a wash buffer, ELISA goat antibodies against human inumunoglobin classes are released from a reagent pouch and incubated with the test pads for up to 30 min, optionally at about 35° C., in an optimized buffer, and the test pads are then rinsed again thoroughly, before chromogenic enzyme substrate is added from a second reagent pouch. After reacting with the enzyme-linked antibody, chromogen typically precipitates on the test pads.

From the plasma lysate in the nucleic acids extraction subcircuit, nucleic acids are captured on a silica fiber filter or similar solid phase bed material. After washing with an alcoholic solution, and drying under blowing air, the nucleic acid retentate, including viral genomic RNA and mRNA, is eluted with elution buffer and transferred to the reverse transcriptase chamber, where, as is well within the skill of those familiar with the art, cDNA first strand copies of the genomic and mRNA species in the lysate are made. Reverse transcriptase reactions are typically run at temperatures between about 30 and 55° C. At least one reverse primer is provided. It is the resulting antisense cDNA copies that are the target of PCR amplification in the next phase of the assay.

In this example, PCR reaction volume is about 50 uL. Representative reaction conditions are 1.7 mM MgCl₂, 0.5 mM deoxynucleoside triphosphate, and 4 U of Platinum Taq DNA polymerase. PCR is performed in a fluidic subcircuit equipped with a variable or two-station fixed temperature thermal interface. Forward and reverse primers are generally dehydrated in the amplification chamber or chambers during manufacture. The number, time, concentrations, and temperature set points of the thermocycling protocol are optimized as is customarily practiced by those skilled in the art.

While various PCR reaction chambers are disclosed herein as elements of the PCR fluidics assembly, the multiplex reactions of the present example are carried out in 4 parallel channels equipped with a dual fixed temperature interface. Each channel utilizes multiple primer pairs. In the current example, 35 thermocycles are performed. Cycle time is about 45 sec, and temperatures of 94° C. for melt and 66° C. for anneal are chosen. By modifying the hardware, or by using timed release solid phase deposits, sequential nested PCR and asymmetric PCR may be performed if desired.

Following amplification, the reactant mixture is mixed with avidin coated magnetic beads. The beads are rehydrated from Rehydration and Wash Buffer Pouch, and mixed by reciprocating flow between the Mag Bead Reservoir and Mixing Chamber of FIG. 4. The bead mixture is then pumped into a detection chamber. The beads, and harvested amplicon, are washed with Rehydration and Wash Buffer while held in place in a magnetic field. The magnetic field is also used to enhance the interaction of the amplicons with the antibodies in the test pad arrays.

The reverse and forward primers are tagged with biotin and a peptide hapten respectively (see FIG. 5). Avidin coated magnetic beads are used to capture biotin-labeled amplicons. Any two-tailed amplicons also containing the hapten-tagged second primer, are then captured on test pads coated with specific antibody to the peptide hapten. In this way, test pads that become colored due to capture of the magnetic bead:two tailed amplicon complexes are consistent with the diagnosis of Measles Virus. By including forward primers for each of the genotypes of the virus, but tagged with individual haptens, a genotype specific diagnosis can be made. The combined information provided from molecular biological testing and immunoassay provide added assurance in the diagnosis and important clinical information about the progress of the infection.

### Example 10

The devices of the present invention are not limited to diagnosis of single pathogens. Panels of multiple pathogens may also be manufactured using the devices described here. For example, a Febrile Panel consisting of means for detecting S. typhi, B. abortus, P. pestis, B. anthracis, B. fragilis, S. pyogenes, and L. pneumophila is designed and fabricated based on the principles disclosed here. These are then packaged in kits. Similarly, a blood sepsis panel or a sexually transmitted disease panel can be designed, fabricated and packaged in kits. The sexually transmitted disease panel can consist of sample processing steps, nucleic acid extraction steps, nucleic acid target amplification steps, and means for detection of Chlamydia trachomatis, Neisseria gonorrhoea, Trichomonas vaginalis, Mycoplasma genitalia, Papilloma Virus, Herpes simplex Virus Type II, and HIV, for example. Obvious variants are contemplated.

### Example 11

Here we take a closer look at a selected application, the screening of biological samples for sexually transmitted diseases. We detail a device (FIG. 6) configured for the simultaneous detection of parasitic protozoa, intracellular parasites, pathogenic bacteria, and viruses which produce venereal disease. Our focus in this example is on the molecular biology. Nucleic acid extraction from a variety of sample types is readily accomplished by lysis and capture of DNA and RNA on solid phase adsorbents. Typical samples are fluidized, but contain a mixture of tissue fluids, whole ruptured host cells, and putative viral, microbial or eukaryotic pathogens and fragments of their DNA and/or RNA and is generally filtered and transferred to a nucleic acid capture matrix for selective adsorption and elution of target nucleic acids.

The eluate from the Nucleic Acid Target Capture Assembly is split immediately in this example into three chambers for special processing. In the first chamber, cDNA Synthesis Chamber 1A (609), antisense primers for Chlamydial mRNA are pre-packaged, along with a reverse transcriptase, dNTPs, 1 U of RNAguard (Pharmacia, St. Albans, Hertsfordshire, United Kingdom), magnesium salts, buffer and other enzymes and cofactors to make cDNA. Tetramethylammonium chloride (TMAC) 0.5 mM is added to improve the fidelity of primer annealing. The products are then pumped into Nested PCR Chamber 1B (620) for further amplification. Within Nested PCR Chamber 1B are forward and reverse primer pairs. In Nested PCR Chamber 1C (621), a second reverse primer, amplifying a nested sequence within the first amplicon product, is ready in dehydrated form, along with all required cofactors to complete the sequential PCR reaction. Note that all chambers are interfaced with variable temperature control elements. The design of the bellows chambers permits reciprocal mixing between pairs of chambers 609 and 620, followed by 620 and 621, for example during the sequential phases of reverse transcription, PCR, and nested PCR.

In the second chamber, cDNA Chamber 2A (610), sense and antisense primers for viral RNA and DNA of HIV, Papilloma Virus, and Herpes simplex Type II, are deposited, along with reverse transcriptase and essential cofactors in a buffered matrix. Upon rehydration with eluate and warming, cDNA copy number is increased significantly, and the reaction mixture is made ready for PCR. Within Nested PCR Chamber 2B (622), 3B (624) and 4B (626) are primers selected for one of the selected pathogens, so that simplex amplification is carried out by thermocycling with reciprocal mixing between Chambers 2B (622), 3B (624) and 4B (626) and Chambers 2C (623), 3C (625) and 4C (627) respectively.

In the third chamber, labelled Nested PCR Chamber 5A (611), forward and reverse primers are used to amplify larger fragments of the genomic DNA of N. gonorrhoea and T. vaginalis. This mixture is transferred to chambers 5B (628) and 6B (630), where nested primers specific to each organism have been deposited. This primer mix is also supplied with fresh TAQ polymerase, and added cofactors needed for multiple rounds of thermocycling. The reaction mixture is mixed by reciprocal pumping between chambers 5B (628) and 5C (629) and between 6B (630) and 6C (631) respectively.

Upon completion of amplification, the amplicons are pumped into detection chambers specific to each target pathogen (Chambers 642-647). In each detection chamber, a FRET probe specific to the target amplicon is used to detect a positive test result, using the fluorescence optoelectronics capability of the host instrument. A positive fluorescence signal, plus the appropriate melt curve, confirms the endpoint determination and thus the STD diagnosis. Optionally, a lateral flow strip with probes forming capture zones could also be used to the same effect.

The card is unique in accommodating a wide variety of samples, from cervical and urethral swabs, to tampons, to synovial fluid, to blood, plasma or serum, and urine sediment, for example. Alone or in combination with immunoassay, the information provided from molecular biological testing panel provide added strength in the diagnostic laboratory capability and can serve to detect co-infections with multiple organisms. Antibody titer is a positive indicator of the prognosis, but molecular tools aid in early identification of infected individuals. Devices of this type are supplied as kits. The kits include the disposable card, which contains all reagents needed for the assay. The user is also provided with a host instrument in which the card is docked during the assay.

While the above description contains specificities, these specificities should not be construed as limitations on the scope of the invention, but rather as exemplifications of embodiments of the invention. That is to say, the foregoing description of the invention is exemplary for purposes of illustration and explanation. Without departing from the spirit and scope of this invention, one skilled in the art can make various changes and modifications to the invention to adapt it to various usages and conditions without inventive step. The breadth of the disclosure is illustrated by the specifications and examples herein, but any patent claims arising from this disclosure are not limited by the literal scope of the specifications and examples contained here.

1-64. (canceled)

65. An apparatus for performing differential laboratory diagnostic testing, comprising:
a) a disposable, single-entry microfluidic card having a plastic body and a sample inlet port for receiving a biological sample, said sample inlet port having a first valved fluidic connection to an immunoassay fluidic subcircuit and a second valved fluidic connection to a nucleic acid assay fluidic subcircuit; and
b) a host instrument having a dock configured for receiving said microfluidic card and a microprocessor with user interface configured for pneumatically controlling a plurality of immunoassays, a plurality of nucleic acid assays, or a combination of assays thereof on said microfluidic card, under control of at least one command entered by a user,
wherein said immunoassay fluidic subcircuit is configured with on-board reagents and means for performing a plurality of immunoassays and said nucleic acid assay fluidic subcircuit is configured with i) on-board reagents, ii) means for performing a plurality of nucleic acid assays, and iii) a nucleic acid extraction subcircuit.

66. The apparatus of claim 65, wherein said biological sample is a blood sample of a vertebrate host and said sample inlet port is configured for filtering said blood sample by aspiration to form a plasma fraction and a cellular fraction, and wherein said plasma fraction is conveyed via said first valved fluidic connection to said immunoassay fluidic sub circuit.

67. The apparatus of claim 66, wherein said cellular fraction is conveyed via said second valved fluidic connection to said nucleic acid extraction subcircuit of said nucleic acid assay fluidic subcircuit.

68. The apparatus of claim 67, further comprising a plasma recycling loop channel fluidly interconnecting said immunoassay fluidic subcircuit and said nucleic acid extraction subcircuit of said nucleic acid assay fluidic subcircuit, whereby said plasma fraction is rejoined with said cellular fraction in said nucleic acid extraction subcircuit.

69. The apparatus of claim 66, wherein said immunoassay fluidic subcircuit comprises a means for detecting an antigen or an antibody, wherein said antigen is associated with an etiological agent of a disease and said antibody is an immune response of said vertebrate host to said etiological agent.

70. The apparatus of claim 66, wherein said means for performing a plurality of immunoassays of said immunoassay fluidic subcircuit comprises a detection chamber, said detection chamber having test pads for immunobinding an antibody or an antigen and on-board reagents for performing an ELISA assay.

71. The apparatus of claim 70, wherein said detection chamber comprises antigen binding test pads and said on-board reagents of said detection chamber are configured for differentiating an IgM antibody from an IgG antibody by ELISA sandwich assay, thereby differentiating an acute phase infection.

72. The apparatus of claim 70 comprising one or more detection chambers for testing an acute fever panel selected from malaria, measles, dengue, rickettsia, salmonella and influenza.

73. The apparatus of claim 71 wherein said antigen binding test pads are configured for detecting malaria-related pan-specific aldolase or type-specific HRP2.

74. The apparatus of claim 65, wherein said on-board reagents of said immunoassay fluidic subcircuit comprise dehydrated antigen, antibody, or enzyme-tagged antibodies.

75. The apparatus of claim 65, wherein said on-board reagents of said nucleic acid assay fluidic subcircuit comprise dehydrated polymerase, primer, reverse transcriptase, molecular beacon, or tagged probe.

76. The apparatus of claim 75, wherein said nucleic acid assay fluidic subcircuit comprises a chamber for first strand cDNA synthesis by reverse transcriptase.

77. The apparatus of claim 75, wherein said nucleic acid assay fluidic subcircuit comprises a chamber for nucleic acid amplification.

78. The apparatus of claim 75, wherein said nucleic acid assay fluidic subcircuit further comprises a thermal-melt FRET detection chamber and said host instrument is configured for ramping the temperature in said FRET detection chamber.

79. The apparatus of claim 75, wherein said nucleic acid assay fluidic subcircuit comprises a plurality of branching parallel pathways configured for simultaneous amplification of more than one nucleic acid assay target.

80. The apparatus of claim 65, wherein said differential laboratory diagnostic testing comprises testing a biological sample for a panel of viruses and bacteria by immunoassay and nucleic acid assay, thereby differentiating a viral infection from a bacterial infection of a vertebrate host.

81. The apparatus of claim 65, wherein said differential laboratory diagnostic testing comprises testing for an acute infection, a chronic infection, or a resolved infection by immunoassay and nucleic acid assay.

82. The apparatus of claim 65, wherein said differential laboratory diagnostic testing comprises testing a blood sample for a nucleic acid of a malarial parasite and an antibody to a malarial antigen.

83. The apparatus of claim 65, wherein said microfluidic card and host instrument are configured for testing a blood sample for viral IgM, or IgM or IgG antibodies by ELISA and for viral nucleic acid by reverse-transcriptase mediated PCR, where the virus is selected from the group consisting of Dengue Virus, Measles Virus, West Nile Virus, Yellow Fever Virus, Equine Encephalitis Virus, HIV or HCV.

84. The apparatus of claim 65, wherein said microfluidic card and host instrument are configured for testing a swab or liquefied solid tissue for a STD panel comprising infectious agents selected from the group consisting of Chlamydia trachomatis, Neisseria gonorrhoea, Trichomonas vaginalis, Mycoplasma genitalia, Papilloma Virus, Herpes simplex Virus Types I or II, HBV, and HIV.


SEQUENCE LISTING (38 sequences)

SEQ ID NO: 1 - DNA, 26 bp, Artificial Sequence
  Primer
tctcgtcagctgacgatctctagtgc

SEQ ID NO: 2 - DNA, 30 bp, Artificial Sequence
  Primer
acgagtgggccctccatcacatttttcttt

SEQ ID NO: 3 - DNA, 19 bp, Artificial Sequence
  Primer
caaagcctccaaatgagga

SEQ ID NO: 4 - DNA, 20 bp, Artificial Sequence
  Primer
tttttggcttctcactctgg

SEQ ID NO: 5 - DNA, 25 bp, Artificial Sequence
  Primer
caatatgctgaaacgcgagagaaac

SEQ ID NO: 6 - DNA, 24 bp, Artificial Sequence
  Primer
ccccatctattcagaatccctgcc

SEQ ID NO: 7 - DNA, 23 bp, Artificial Sequence
  Primer
cgctccatacatcttgaatgagc

SEQ ID NO: 8 - DNA, 21 bp, Artificial Sequence
  Primer
aagacattgatggcttttgac

SEQ ID NO: 9 - DNA, 22 bp, Artificial Sequence
  Primer
aagacgtaaatagcccccgacc

SEQ ID NO: 10 - DNA, 24 bp, Artificial Sequence
  Primer
aggactcgcaaaaacgtgataatc

SEQ ID NO: 11 - DNA, 18 bp, Artificial Sequence
  Primer
gccatatggtacatgtgg

SEQ ID NO: 12 - DNA, 21 bp, Artificial Sequence
  Primer
tgtcccatcctgcggtatcat

SEQ ID NO: 13 - DNA, 22 bp, Artificial Sequence
  Primer
gggtgtccgtgtctgagccttg

SEQ ID NO: 14 - DNA, 23 bp, Artificial Sequence
  Primer
gcaatgcatactactgaggacaa

SEQ ID NO: 15 - DNA, 21 bp, Artificial Sequence
  Primer
caggacagtcgaaggtcagcc

SEQ ID NO: 16 - DNA, 25 bp, Artificial Sequence
  Primer
cgagatgggggggtaaggaagatat

SEQ ID NO: 17 - DNA, 24 bp, Artificial Sequence
  Primer
gatcaaagtgagaatgagctccca

SEQ ID NO: 18 - DNA, 24 bp, Artificial Sequence
  Primer
gatcaaagtgagaatgagctacca

SEQ ID NO: 19 - DNA, 24 bp, Artificial Sequence
  Primer
gatcaaagtgggagtgagctacca

SEQ ID NO: 20 - DNA, 19 bp, Artificial Sequence
  Primer
ccgggcacagcagagcaaa

SEQ ID NO: 21 - DNA, 21 bp, Artificial Sequence
  Primer
cattgacactgcatcggagta

SEQ ID NO: 22 - DNA, 20 bp, Artificial Sequence
  Primer
acagtcgaaggtcagccgat

SEQ ID NO: 23 - DNA, 21 bp, Artificial Sequence
  Primer
aggacaggagggtcaaacagg

SEQ ID NO: 24 - DNA, 21 bp, Artificial Sequence
  Primer
gagaaaccgggtccagcagaa

SEQ ID NO: 25 - DNA, 25 bp, Artificial Sequence
  Primer
cccagacaagcccaagtgtcattta

SEQ ID NO: 26 - DNA, 26 bp, Artificial Sequence
  Primer
cctagacattgacactgcatcggaga

SEQ ID NO: 27 - DNA, 24 bp, Artificial Sequence
  Primer
gtcaaacagagtcggggagaagca

SEQ ID NO: 28 - DNA, 21 bp, Artificial Sequence
  Primer
ctgcaagccatggcaggaatc

SEQ ID NO: 29 - DNA, 21 bp, Artificial Sequence
  Primer
gcccatcctccaaccagcatg

SEQ ID NO: 30 - DNA, 21 bp, Artificial Sequence
  Primer
ggtatcactgccgaggatgcg

SEQ ID NO: 31 - DNA, 23 bp, Artificial Sequence
  Primer
ccaagatctgcaggacagccgac

SEQ ID NO: 32 - DNA, 20 bp, Artificial Sequence
  Primer
gggagaagccagggagagca

SEQ ID NO: 33 - DNA, 21 bp, Artificial Sequence
  Primer
ctgcaagccatggcaggaatc

SEQ ID NO: 34 - DNA, 22 bp, Artificial Sequence
  Primer
gcaaatgatgcgagagctgctg

SEQ ID NO: 35 - DNA, 28 bp, Artificial Sequence
  Primer
cgggattggggggtaaggaagataagaa

SEQ ID NO: 36 - DNA, 24 bp, Artificial Sequence
  Primer
ccaggcaagcccaagtctcatttt

SEQ ID NO: 37 - DNA, 21 bp, Artificial Sequence
  Primer
ctacagagaaaccgggctcaa

SEQ ID NO: 38 - DNA, 18 bp, Artificial Sequence
  Primer
ttataacaatgatggagg